(19) United States
(12) Patent Application Publication
(10) Pub. No.: US 2011/0076734 A1
(43) Pub. Date: Mar. 31, 2011

(54) Electrowetting Microarray Printing System and Methods for Bioactive Tissue Construct Manufacturing

(75) Inventors: Gongyao Zhou, Cherry Hill, NJ (US); Peter I. Lelkes, Cherry Hill, NJ (US); Robert B. Fair, Durham, NC (US)

(73) Assignees: DREXEL UNIVERSITY; DUKE UNIVERSITY

(21) Appl. No.: 12/921,333

(22) PCT Filed: Mar. 6, 2009
(86) PCT No.: PCT/US09/36371

§ 371 Date: Nov. 29, 2010

Related U.S. Application Data
(60) Provisional application No. 61/068,501, filed on Mar. 7, 2008.

(51) Int. Cl. C12N 13/00; C12M 3/00

(52) U.S. Cl. 435/173.1; 435/283.1

(57) ABSTRACT

Apparatuses and methods for manufacturing three-dimensional, bioactive, tissue scaffold fabrications with embedded cells and bioactive materials, such as growth factors, using biomimetic structure modeling, solid freeform fabrication, biocompatible hydrogel material, and electrowetting on dielectric-based multi-microarray printing.

### STATEMENT REGARDING FEDERALLY SPONSORED RESEARCH OR DEVELOPMENT

This invention was made, in part, using funds obtained from the U.S. Government (National Science Foundation Award No. 0700139) and the U.S. Government therefore has certain rights in this invention.

### BACKGROUND OF THE INVENTION

Tissue Engineering (TE) is evolving as a potential solution for the repair and reconstruction of diseased or damaged tissues (Langer and Vacanti, 1993, Science, 260:920-926). In the US alone, about eight million surgical procedures are performed each year to treat tissue-related maladies. Furthermore, over 70,000 patients are waiting for the organs to be donated, and more than 100,000 people die with tissue related disorders.

A variety of methods have been developed for manufacturing 3D scaffolds with embedded cells and growth factors for soft tissue engineering. These methods can be generally classified as two categories: non-automation methods and Solid Freeform Fabrication (SFF). Non-automation methods have been used for simple research purposes, while computer-aided SFF has been used to better control scaffold architecture with cells and growth factors embedded.

Non-automation methods used to manufacture scaffolds fall into several general classes. First, cells can be cultured and attached to flat surfaces, where they will synthesize extracellular matrix (ECM). Upon reaching confluence, a sheet of cells and ECM can be peeled from the substrate and rolled to form a tubular blood vessel media (L'Heureux et al., 1998, FASEB J, 12:47-56) or stacked to form a thicker block of tissue (Baar et al., 2005, FASEB J, 19:275). Second, cells can be mixed with ECM proteins such as collagen or Matrigel, or with hydrogel precursors that are allowed to gel with cells entrapped. Gels can then be compacted and shaped by a mechanical means such as centrifugation. Third, porous polymer scaffolds can be formed in bulk and then seeded with cells. Typical bulk synthetic scaffolds can be fibrous or cellular. Fibrous scaffolds can be woven or nonwoven mats, and can be preferentially oriented by strain or centrifugal forces. Cellular scaffolds can be formed by porogen leaching, gas foaming, gel casting, solution casting and gel freeze drying, among other methods. In each case, cells are seeded onto the prepared scaffold. Fourth, a cellularized tissue such as small intestinal submucosa can be used as a non-synthetic scaffold material and then seeded with cells.

Non-automation methods have the advantages of simplicity and low cost, but the control over the microarchitecture is limited to approximate control of pore size for cast hydrogels, and fiber size and planar orientation (linear or random mats) for fibrous networks. Further, control of seeding is limited to cell density control and heterogeneous cell patterning cannot be achieved. In most non-automation methods, an internal porous structure is generated by randomly packed porogen and cannot be controlled precisely or flexibly. For example, the pore size and porosity at different sections of the scaffold should be different in many cases, and all the pores should be interconnected; however, these requirements cannot be obtained or guaranteed. The biggest limitation with these methods is their incapability for making complex 3D multicellular constructs, as well as their incapability for incorporating a vascular network.

SFF is a newer manufacturing technology, involving a group of technologies that are together capable of producing complex freeform parts directly from a computer aided design (CAD) model of an object, without part-specific tooling or fixture. The CAD model can be designed using 3D CAD software or obtained through reverse engineering, through the reconstruction of three dimension models from the data produced by Computed Tomography (CT), Magnetic Resonance Imaging (MRI) or a 3D coordinate measuring machine. The CAD model is then transferred into sliced layers. Based on these layers, numerical control codes are generated to control the machine in building the part.

SFF possesses several unique advantages that make it a powerful manufacturing tool for 3D scaffolds. First, a three-dimensional CAD model of the tissue can be reconstructed precisely by reverse engineering software based on the data of CT or MRI system. The SFF machine can subsequently make the scaffold with any complex geometry. Second, SFF technology makes parts in an additive fashion through a layer-by-layer process. In each layer, materials can be added line by line, even dot by dot, so the internal structure of the porous scaffold can be controlled directly and precisely to meet any special requirements, including relatively complex and curved shapes such as myocardial microvascular network. Third, a wide range of biomaterials of hydrogels are available to make scaffolds for soft tissue using SFF technology.

SFF methods look promising for manufacturing scaffolds for bone tissue engineering (Yeong et al. 2004, Trends in Biotechnology, 22), but the potential of SFF has not yet been fully exploited for soft tissue engineering yet. Of several SFF methods being studied, the most popular uses commercially available inkjet printers, with slight modifications, to print solutions of cells and of growth factors, with a resolution as small as 200 μm. Also, extrusion-based SFF techniques have been reported for making hydrogel scaffolds with embedded cells, which are dispensed in lines, deposited in parallel and layer by layer to create 3D tissue constructs with grid-like architecture. Also, a pulsed-laser-based printing technique has been reported for printing individual cells from a slide coated with a laser-absorptive layer. This method has some advantages, such as its ability to print single cells and to print very small size of droplets (100 nm-10 μm) (Barron et al., 2005, Ann Biomed Eng 33:121-130). Also, photopolymerisable hydrogels have been used in combination with the computer aided methods to create cell encapsulated hydrogel scaffolds.

Many types of micro-droplet generators that do not control surface tension (ST) have been used for bio-engineering applications. Each type has its own advantages and limitations (see FIG. 9). Liquid handling and actuation by controlling ST has many advantages in microscale applications because of the dominance and effectiveness of the ST force, as the liquid handling system becomes smaller.

One tool for manipulating tiny amounts of liquids on surfaces based on ST manipulation is Electrowetting On Dielectric (EWOD) (see U.S. Pat. Nos. 6,989,234 and 6,911,132). EWOD has not been used in TE. Current EWOD applications include biomedical diagnostics, adjustable lenses, ‘lab-on-a-chip’ systems for applications such as DNA and protein analysis, and new kinds of electronic displays (Cho et al., 2003, J Microelectromechanical Systems 12:70-80; Fair et al., 2003, IEEE International Electronic Devices Meeting, pp. 32.5.1-32.5.4).

EWOD uses the electrocapillary principle: ST is a function of electric potential across an interface, and the change in liquid-solid ST, γ_{SL}, changes the contact angle at the liquid-solid-gas interfaces (FIG. 1). When voltage, V, is applied between the liquid and electrode, surface energy is balanced by electrical energy, and γ_{SL} is lowered, as expressed in Lippmann's equation (FIG. 10, equation 1). Young's (equation 2) relates contact angle and ST, and Lippmann-Young's (equation 3) relates contact angle to voltage (Cho et al., 2002, The Fifteenth IEEE International Conference on Micro Electro Mechanical Systems, pp. 32-35). In EWOD, a droplet of liquid rests on a surface or in a channel coated with a hydrophobic, dielectric material. Charge accumulates at the solid-liquid interface, and the surface wettability is modified from hydrophobic to hydrophilic by applying a voltage between the liquid and an electrode under the dielectric layer. EWOD uses the change in contact angle direction to induce liquid motions. Droplets are ejected from reservoirs and programmed to move to specific locations where they can be merged and cut. By applying a sequence of voltage to electrodes patterned under the dielectric layer, four fundamental droplet manipulation mechanisms can be established (FIG. 2): (1) creating, (2) cutting, (3) joining, and (4) transporting of droplets from a reservoir and in the fluid path.

All of the SFF methods for soft TE are at an early stage of development and continue to face many challenges. Inkjet printing methods depend on commercial inkjets, which were designed to dispense ink. These systems can function only in a narrow, low viscosity range, which limits the type and strength of solutions that can be printed. In addition, inkjet printers have problems with cells clogging the jets, have a resolution limited to about 200 μm and are not well-suited for dispensing living cells, based on the 25% cell death that has been reported (Wilson & Boland, 2003, Anat Rec A Discov Mol Cell Evol Biol 272:491-496). This process also includes an extra step of using an intermediate substrate “bio paper” to support cells, because the process cannot jet out hydrogel along with cells, which limits the prospects of this method for 3D printing.

Extrusion-based SFF methods produce a limited range of scaffold architectures, with parallel linear elements stacked in layers, at a resolution of around 100 μm, and do not enable heterogeneous cell patterning (precise arrangement of multiple cell types). Laser-based SFF methods expose cells to high stress, UV light, and heat, which must be carefully controlled to avoid damaging cells. SFF methods also may not scale up easily to 3D manufacturing because cells are usually delivered from 2D arrays. Although TE has recently been used successfully to replace a blood vessel in pediatric surgery (Yuji, 2001, J Thorac Cardiovasc Surg Volume 125:419-420), TE is still relatively new and has a long way to go to effectively and efficiently manufacture biocompatible and bioactive tissue substitute. To become effective and efficient, TE must overcome several challenges. First, manufacturing techniques that mimic tissue and extra cellular matrix (ECM) architecture, with high resolution (less than 10 μm) for tissues such as, for example, myocardium (heart muscle), blood vessels, bone or nerves are needed. Second, innovative multiple-jet printing methods for the delivery of cells and growth factors into scaffolds are needed. Third, manufacturing techniques for building complex structures (e.g., vascular structure) enabling nutrient transport are needed. The current invention fulfills these needs.

### SUMMARY OF THE INVENTION

The invention disclosed herein relates to apparatuses and methods for engineering tissue using electrowetting techniques. The apparatuses and methods of the invention allow for the manufacturing of tissue by the deposition of a variety of biomaterials with a single innovative tissue manufacturing system. The invention includes a system and methods for manufacturing 3D, bioactive, tissue scaffold fabrications with embedded cells and bioactive materials, such as growth factors, using biomimetic structure modeling, SFF, biocompatible hydrogel material, and EWOD-based multi-microarray printing.

In one embodiment, the invention is an apparatus for engineering tissue by actuating droplets using electrowetting techniques. In various embodiments, the apparatus of the invention can have at least four microarray print heads, but can also have at least two, or three, or more than four print heads. Each of the microarray print heads has a first conductive layer having an array of control electrodes covered by a first hydrophobic insulator surface; a second conductive layer having a second conductive layer surface facing the first hydrophobic surface, the second conductive layer spaced from the first conductive layer to define a gap there between, and having an actuation voltage thereon of 20 to 100 volts; and a wire traction system having at least one conductive elongate wire element disposed in the gap between the first and second conductive layers and having a second hydrophobic surface, and having a voltage thereon less than the second conductive layer actuation voltage; and a voltage source communicating with the second conductive layer and the elongate wire element that provides an actuation voltage to the second conductive layer of 20 to 100 volts, wherein the droplet is caused to move along a pathway extending around the conductive elongate wire element and from the first conductive layer towards the second conductive layer. In embodiments of the invention having at least four microarray print heads, one of the print heads actuates droplets containing a hydrogel, one of the print heads actuates droplets containing a crosslinker, one of the print heads actuates droplets containing a cell suspension, and one of the print heads actuates droplets containing a growth factor.

In another embodiment of the apparatus for engineering tissue by manipulating droplets, each of the microarray print heads has a substrate having a substrate surface; an array of drive electrodes disposed on the substrate surface; a dedicated array of reference elements settable to a common reference potential and disposed in at least substantially co-planar relation to the electrode array, wherein the array of reference elements is electrically and physically distinct from the drive electrode array and further wherein each drive electrode is adjacent to at least one of the reference elements; a dielectric layer disposed on the substrate surface to cover the drive electrodes; and an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes. In embodiments of the invention having at least four microarray print heads, one of the print heads actuates droplets containing a hydrogel, one of the print heads actuates droplets containing a crosslinker, one of the print heads actuates droplets containing a cell suspension, and one of the print heads actuates droplets containing a growth factor.

In still another embodiment of the apparatus for engineering tissue by manipulating droplets, each of the microarray print heads has a substrate having a substrate surface; an array of electrodes disposed in at least substantially co-planar relation on the substrate surface, wherein the array of electrodes comprises drive electrodes and dedicated reference electrodes; a dielectric layer disposed on the substrate surface and covering the array of electrodes; an electrode selector for dynamically creating a sequence of electrode pairs, each electrode pair having a selected one of the drive electrodes biased to a first voltage and a selected one of the reference electrodes disposed adjacent to the selected drive electrode and biased to a second voltage less than the first voltage, whereby a droplet disposed on the substrate surface moves along a desired path running between the electrode pairs created by the electrode selector; and whereby manipulation of the droplet is accomplished by electrowetting actuation wherein the droplet overlaps a selected one of the drive electrodes and a selected one of the reference electrodes continuously. In embodiments of the invention having at least four microarray print heads, one of the print heads actuates droplets containing a hydrogel, one of the print heads actuates droplets containing a crosslinker, one of the print heads actuates droplets containing a cell suspension, and one of the print heads actuates droplets containing a growth factor.

In yet another embodiment, the invention is a method of tissue engineering by actuating droplets by electrowetting, comprising the steps of horizontally actuating a first group of droplets containing a hydrogel to position each droplet of the first group of droplets at a discrete target location on a microfluidic chip, and vertically actuating the first group of droplets to deposit the first group of droplets onto a tissue growth surface; horizontally actuating a second group of droplets containing a crosslinker to position each droplet of the second group of droplets at a discrete target location on a microfluidic chip and vertically actuating the second group of droplets to deposit the second group of droplets onto the tissue growth surface; horizontally actuating a third group of droplets containing a cell suspension to position each droplet of the third group of droplets at a discrete target location on a microfluidic chip vertically actuating the third group of droplets to deposit the third group of droplets onto the tissue growth surface; horizontally actuating a fourth group of droplets containing a growth factor to position each droplet of the fourth group of droplets at a discrete target location on a microfluidic chip and vertically actuating the fourth group of droplets to deposit the fourth group of droplets onto the tissue growth surface. In various embodiments of the methods of the invention, the methods steps described herein are repeated to deposit a subsequent layer of droplets onto the tissue growth surface to engineer the desired tissue.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings. In the drawings:

FIG. 1 depicts the principle of EWOD. In EWOD, a droplet of liquid rests on a surface or in a channel coated with a hydrophobic, dielectric material. Charge accumulates at the solid-liquid interface, and the surface wettability is modified from hydrophobic to hydrophilic by applying a voltage between the liquid and an electrode under the dielectric layer. EWOD uses the change in contact angle direction to induce liquid motions.

FIG. 2 depicts fundamental droplet operations. Droplets are ejected from reservoirs and programmed to move to specific, discrete target locations where they can be positioned, or merged, or cut. By applying a sequence of voltage to electrodes patterned under the dielectric layer, four fundamental droplet manipulation mechanisms can be established (FIG. 2): (1) creating, (2) cutting, (3) joining, and (4) transporting of droplets from a reservoir and in the fluid path.

FIG. 3 depicts the architecture of an array printing system.

FIG. 4 depicts a schematic of an EWOD-based multi-microarray printing system.

FIG. 5 depicts high viscosity (250 centipoise (cP)) droplet manipulation.

FIG. 6 depicts hydrogel formation on a chip.

FIG. 7 depicts cells after EWOD actuation. The fraction of dead cells (indicated by arrows) after actuation was comparable to the fraction of dead cells before actuation.

FIG. 8 depicts a base block and top layer.

FIG. 9 depicts a comparison of the advantages and disadvantages of different micro-droplet generator methods.

FIG. 10 depicts design equations.

FIG. 11 depicts a cross-sectional view of an electrowetting microactuator mechanism having a two-sided electrode configuration.

FIG. 12 depicts a top plan view of an array of electrode cells having interdigitated perimeters.

FIG. 13, comprising FIGS. 13A-13D, depicts sequential schematic views of a droplet being moved by the electrowetting technique of the present invention.

FIG. 14, comprising FIGS. 14A-14C, depicts sequential schematic views illustrating two droplets combining into a merged droplet using the electrowetting technique of the present invention.

FIG. 15, comprising FIGS. 15A-15C, depicts sequential schematic views showing a droplet being split into two droplets by the electrowetting technique of the present invention.

FIG. 16, comprising FIGS. 16A and 16B, depicts sequential schematic views showing a liquid being dispensed on an electrode array and a droplet being formed from the liquid.

FIG. 17, comprising FIGS. 17A and 17B, depicts a cross-sectional view of an electrowetting microactuator mechanism having a single-sided electrode configuration in accordance with another embodiment of the present invention (FIG. 17A) and a top plan view of a portion of the mechanism depicted in FIG. 17A with its upper plane removed (FIG. 17B).

FIG. 18, comprising FIGS. 18A-18D, depicts sequential schematic views of an electrowetting microactuator mechanism having an alternative single-sided electrode configuration, illustrating electrowetting-based movement of a droplet positioned on a misaligned electrode array of the mechanism.

FIG. 19, comprising FIGS. 19A and 19B, depicts schematic views of an alternative electrowetting microactuator mechanism having a single-sided electrode configuration arranged as an aligned array, respectively illustrating a droplet actuated in north-south and east-west directions.

FIG. 20, comprising FIGS. 20A and 20B, depicts a side elevation view in cross-section of a droplet actuating apparatus provided in accordance with the present invention, wherein a droplet has been placed on a first plane of the apparatus (FIG. 20A) and a side elevation view in cross-section of the apparatus depicted in FIG. 20A, wherein the droplet has been actuated into contact with a second plane spaced from the first plane (FIG. 20B).

FIG. 21 depicts a side elevation view in cross-section of a droplet actuating apparatus provided in accordance with an alternative embodiment of the present invention.

FIG. 22, comprising FIGS. 22A and 22B, depicts a side elevation view in cross-section of an encapsulated droplet actuating apparatus provided in accordance with the present invention (FIG. 22A) and an exploded perspective view of the encapsulated droplet actuating apparatus depicted in FIG. 22A (FIG. 22B).

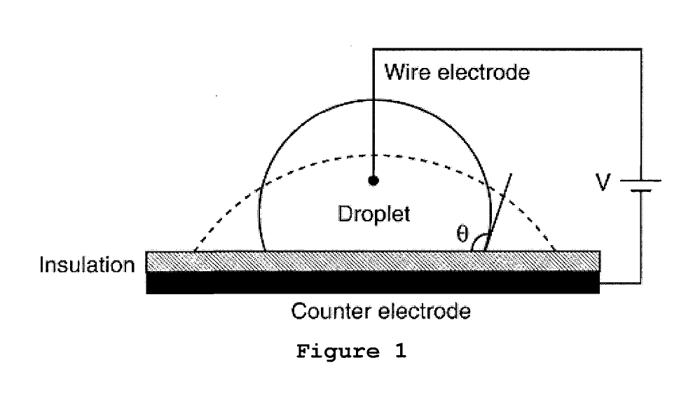

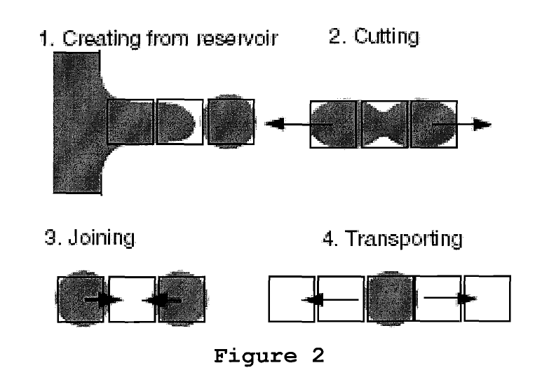

### DETAILED DESCRIPTION OF THE INVENTION

The invention disclosed herein overcomes the existing limitations for fabricating bioactive tissue scaffolds, by combining the advantages of the SFF method with a new application of electrowetting-based microstructure printing, permitting the deposition of various biomaterials with a single innovative tissue manufacturing system. The invention includes a system and methods for manufacturing 3D, bioactive, soft-tissue scaffold fabrications with embedded cells and bioactive materials, such as growth factors, using biomimetic structure modeling, SFF, biocompatible hydrogel material, and EWOD-based multi-microarray printing. In some embodiments, the system and methods of the invention integrate an EWOD array design with a computer-controlled motion system for positioning the EWOD chip for the delivery of hydrogel, crosslinker, cells, and bioactive materials, such as growth factors, on CAD models. The inventive system employs the principle of EWOD multiple microfluidics array printing, which is capable of creating and depositing droplets with sizes of less than about 10 μm, can work with a variety of hydrogels, and can deposit cells and bioactive materials, such as growth factors, during scaffold fabrication.

### Scaffolds

For engineering soft tissues, scaffolds can comprise any suitable synthetic or natural biopolymer that can provide a porous (from 50% up to greater than 90%) support structure, thus mimicking the natural ECM environment in which cells attach, multiply, migrate and function (see Zeltinger et al., 2001, Tissue Engineering 7:557-572). In some embodiments, scaffolds can be resorbable by the body at a rate that is similar to the rate that cells can produce their own natural ECM. In preferred embodiments, scaffolds suitable for use in the invention can provide mechanical support during the body's reconstruction process, can maintain the initially fabricated 3D shape, and can withstand handling during implantation and in vivo loading. In some embodiments, the pores of the scaffold can be interconnected to allow both the ingrowth of cells and the transport of nutrients. The transport of nutrients through the interconnected pores of the scaffold can occur passively or actively. In some embodiments, the scaffold can be biocompatible. In other embodiments, the scaffold can contain bioactive materials, such as growth factors that can, for example, support living cells or enhance new tissue ingrowth. In some embodiments, the scaffold can contain cells that will secrete new ECM that is bio-mechanically similar to the tissue-specific ECM that will be replaced by the body.

The scaffold can be manufactured to any suitable, germane, complex three-dimensional shape, that is similar in size and shape of the tissue or organ to be replaced, at both the microscopic and macroscopic levels. At the cell scale (e.g., on the order of about 10 μm), the microarchitecture of the scaffold pores or fibers can be designed to control cell orientation and migration, which can ultimately affect cell function. At a slightly larger scale (e.g., on the order of about 100 μm), many tissues would require a microvascular-type network to function optimally after implantation, can be designed to be present at the time of manufacture and implantation (see Yin et al., 2004, Am J Physiol Heart Circ Physiol 287:H1276-1285). Currently, most TE constructs do not include vascular networks and thus vascular ingrowth can only occur after implantation, a feature which limits the size and cellular content of implants, and can delay integration with the body. At the tissue scale, the overall size and shape can be designed to be similar to the tissue or organ to be replaced.

In preferred embodiments, scaffolds include hydrogels. Hydrogels are a class of highly hydrated polymer materials with water content over 30% by weight that can be made from either natural or synthetic components. Hydrogels have been widely used in various biomedical applications including TE, due to their biocompatibility, low toxicity and low cost. Hydrogels have excellent mechanical properties that are similar those of soft tissues. Hydrogel molecules can be modified or functionalized, to, by way of nonlimiting examples, promote cell proliferation, cell migration and cell adhesion. The pore size of the gel network can be designed to allow for optimal diffusion and transport of biological molecules (see Watanabe et al., 2004, Hydrogels, in Encyclopedia of Biomaterials and Biomedical Engineering). Further, hydrogels are highly permeable, which can facilitate the exchange of oxygen and other dissolved gases, nutrients, water-soluble metabolites, and the like. The hydrophilicity of some hydrogels prevents protein adsorption, which serves to minimize foreign body responses when implanted in vivo.

One non-limiting example of a hydrogel useful as a scaffold material is chitosan hydrogel. Chitosan is a natural polymer. Chitosan is a deacetylated derivative of chitin commonly found in the shells of crustaceans. One advantage of using a natural polymer, such as chitosan, is that they are generally more likely to mimic the ECM and provide bio-inductive properties desirable for tissue engineering. Chitosan has been extensively used in drug delivery and tissue engineering because of its biocompatibility, biodegradability, antibacterial properties, bioadhesion and low cost (see Adekogbe & Ghanem, 2005, Biomaterials, 2005. 26: p. 7241-7250). Further, chitosan is degraded by lysozyme, an enzyme present naturally in the human body, allowing resorption of the material during subsequent tissue replacement.

Chitosan can be combined with additional biomaterials such as, by way of non-limiting examples, alginate, collagen, gelatin, chitin, hydroxyapatite, PMMA, calcium phosphate cement, b-tricalcium phosphate, poly-lactic-co-glycolic acid (PLGA). The a variety of bioactive materials, such as, for example, growth factors, can be included in the scaffolds useful in the invention. Of the many possible additional bioactive materials, collagen-I is known to be secreted by cardiac fibroblasts in the ventral myocardium and appears to be a good choice for use in cardiac tissue engineering. Although the use of chitosan in the context of cardiac tissue engineering is novel, similar studies have demonstrated the feasibility of culturing fetal cardiac cells in vivo on a 3D alginate scaffold (See Leor et al., 2000, Circulation 102:56-61). Further, it has been demonstrated that a PGL scaffold seeded with fibroblasts can induce angiogenesis around damaged cardiac tissue in mice (see Kellar et al., 2001, Circulation 104:2063).

### Hydrogel Materials and Crosslinkers

In the multi-jet printing process described herein, a hydrogel can be solidified (i.e., gelated) in a variety of ways known to those skilled in the art. By way of non-limiting examples, a hydrogel can be solidified by adding a crosslinker (i.e., crosslinking agent) after the hydrogel is deposited, or by photocrosslinking using, for example, UV light, or by using a thermoresponsive polymer.

In some embodiments, the hydrogel can be crosslinked using a variety of crosslinkers, including covalent or ionic crosslinkers. Covalently crosslinked hydrogels are bound by irreversible chemical links (see Berger et al., 2004, Eur J Pharmaceutics and Biopharmaceutics 57:19-34). By way of a non-limiting example, Genipin is a crosslinker useful in the invention. Genipin is a naturally occurring covalent crosslinking reagent which has been used in herbal medicine and food dyes, has been used as crosslinker of chitosan with little or no cytotoxic effect. In some embodiments, chitosan can be used as the hydrogel material and genipin can be used as the crosslinker. In other embodiments, ionic crosslinkers, such as, for example, tripolyphosphate (which has been used for simulation of permeability of drugs through skin), can be used. As one of skill in the art would understand, it is important to use care in selecting an appropriate crosslinker, as some have been reported to be relatively cytotoxic.

In other embodiments, the hydrogel can be crosslinked by using photocrosslinking by way of for example, the use of a photoinitiator. Photoinitiators have been used for the UV curing of systems comprising unsaturated monomers and prepolymers. Irgacure®2959, for example, has been used mixed with ethanol to create a photoinitiator stock. A photoinitiator stock can be mixed with a variety of different kinds of solutions to be gelled. Poly(ethylene glycol)-diacrylate (PEG-DA) dissolved in phosphate buffered saline (PBS), for example, has been mixed with a photoinitiator stock to create a hydrogel mixture. Also, PEG-Fibrinogen, for example, can be used to create a hydrogel that possess improved cell adhesion properties. (see, for example, Ciba® IRGACURE® 2959)

In still other embodiments, gelation can be achieve through using a thermoresponsive polymer, such as one that forms a free flowing solution in water at ambient temperatures (for example, at room temperature), and a gel at higher temperatures (for example, at body temperature). Such a thermoresponsive hydrogel solution undergoes a phase transformation at its lower critical solution temperature (LCST) to either form a gel or aqueous solution. An example of such a polymer is poly(N-isopropylacrylamide) (PNIPAAm). Another example of such a polymer, poly(ethylene glycol)dimethacrylate (PEGDM), was added to create a branched copolymer. Addition of a branching copolymer at varied ratios can be used to adjust the flexibility or rigidity of the hydrogel to achieve the desired properties (for example, a hydrogel that can better withstand manipulation). (See, for example, Vernengo, 2007, Journal of Biomedical Materials Research Part B: Applied Biomaterials, 23:653).

### Droplet-Based Actuation by Electrowetting

The present invention provides droplet-based handling and manipulation methods by implementing electrowetting-based techniques. The droplets can be moved by controlling voltages to electrodes. Generally, the actuation mechanism of the droplet is based upon surface tension gradients induced in the droplet by the voltage-induced electrowetting effect. The mechanisms of the invention allow the droplets to be transported. The chip can include an array of electrodes that are reconfigurable in real-time to perform desired tasks. The invention enables several different types of handling and manipulation tasks to be performed on independently controllable droplet samples.

Methods of the present invention form droplets for independently transporting, merging, mixing, and other processing of the droplets. The preferred embodiment uses electrical control of surface tension (i.e., electrowetting) to accomplish these manipulations. In one embodiment, the droplet is contained within a space between two parallel plates. One plate contains etched drive electrodes on its surface while the other plate contains either etched electrodes or a single, continuous plane electrode that is grounded or set to a reference potential. Hydrophobic insulation covers the electrodes and an electric field is generated between electrodes on opposing plates. This electric field creates a surface-tension gradient that causes a droplet overlapping the energized electrode to move towards that electrode. Through proper arrangement and control of the electrodes, a droplet can be transported by successively transferring it between adjacent electrodes. The patterned electrodes can be arranged in a two dimensional array so as to allow transport of a droplet to any location covered by that array. The space surrounding the droplets may be filled with a gas such as air or an immiscible fluid such as oil.

In another embodiment, the structure used for ground or reference potential is co-planar with the drive electrodes and the second plate, if used, merely defines the containment space. The co-planar grounding elements can be a conductive grid superimposed on the electrode array. Alternatively, the grounding elements can be electrodes of the array dynamically selected to serve as ground or reference electrodes while other electrodes of the array are selected to serve as drive electrodes.

Droplets can be combined together by transporting them simultaneously onto the same electrode. Droplets are subsequently mixed either passively or actively. Droplets are mixed passively by diffusion. Droplets are mixed actively by moving or “shaking” the combined droplet by taking advantage of the electrowetting phenomenon. In a preferred embodiment, droplets are mixed by rotating them around a two-by-two array of electrodes. The actuation of the droplet creates turbulent non-reversible flow, or creates dispersed multilaminates to enhance mixing via diffusion. Droplets can be split off from a larger droplet or continuous body of liquid in the following manner: at least two electrodes adjacent to the edge of the liquid body are energized along with an electrode directly beneath the liquid, and the liquid moves so as to spread across the extent of the energized electrodes. The intermediate electrode is then de-energized to create a hydrophobic region between two effectively hydrophilic regions. The liquid meniscus breaks above the hydrophobic regions, thus forming a new droplet.

According to one embodiment of the present invention, an apparatus for manipulating droplets comprises a substrate comprising a substrate surface, an array of electrodes disposed on the substrate surface, an array of reference elements, a dielectric layer disposed on the substrate surface, and an electrode selector. The reference elements are settable to a reference potential. The array of reference elements is disposed of in substantially co-planar relation to the electrode array, such that each reference element is adjacent to at least one of the electrodes. The dielectric layer is disposed on the substrate surface and is patterned to cover the electrodes. The electrode selector can be provided as a microprocessor or other suitable component for sequentially activating and de-activating one or more selected electrodes of the array to, sequentially bias the selected electrodes to an actuation voltage. The sequencing performed by the electrode selector enables a droplet disposed on the substrate surface to move along a desired path that is defined by the selected electrodes.

According to one method of the present invention, a droplet is actuated by providing the droplet on a surface that comprises an array of electrodes and a substantially co-planar array of reference elements. The droplet is disposed on a first one of the electrodes, and at least partially overlaps a second one of the electrodes and an intervening one of the reference elements disposed between the first and second electrodes. The first and second electrodes are activated to spread at least a portion of the droplet across the second electrode. The first electrode is de-activated to move the droplet from the first electrode to the second electrode.

According to one aspect of this method, the second electrode is adjacent to the first electrode along a first direction. In addition, the electrode array comprises one more additional electrodes adjacent to the first electrode along one or more additional directions. The droplet at least partially overlaps these additional electrodes as well as the second electrode. In accordance with this aspect of the method, the first direction that includes the first electrode and the second electrode is selected as a desired direction along which the droplet is to move. The second electrode is selected for activation based on the selection of the first direction.

In accordance with another method of the present invention, a droplet is split into two or more droplets. A starting droplet is provided on a surface comprising an array of electrodes and a substantially co-planar array of reference elements. The electrode array comprises at least three electrodes comprising a first outer electrode, a medial electrode adjacent to the first outer electrode, and a second outer electrode adjacent to the medial electrode. The starting droplet is initially disposed on at least one of these three electrodes, and at least partially overlaps at least one other of the three electrodes. Each of the three electrodes is activated to spread the starting droplet across the three electrodes. The medial electrode is de-activated to split the starting droplet into first and second split droplets. The first split droplet is disposed on the first outer electrode and the second split droplet is disposed on the second outer electrode.

In yet another method of the present invention, two or more droplets are merged into one droplet. First and second droplets are provided on a surface comprising an array of electrodes in a substantially co-planar array of reference elements. The electrode array comprises at least three electrodes comprising a first outer electrode, a medial electrode adjacent to the first outer electrode, and a second outer electrode adjacent to the medial electrode. The first droplet is disposed on the first outer electrode and at least partially overlaps the medial electrode. The second droplet is disposed on the second outer electrode and at least partially overlaps the medial electrode. One of the three electrodes is selected as a destination electrode. Two or more of the three electrodes are selected for sequential activation and de-activation, based on the selection of the destination electrode. The electrodes selected for sequencing are sequentially activated and de-activated to move one of the first and second droplets toward the other droplet, or both of the first and second droplets toward each other. The first and second droplets merge together to form a combined droplet on the destination electrode.

According to one aspect of this method, the first droplet comprises a first composition, the second droplet comprises a second composition, and the combined droplet comprises both the first and second compositions. The method further comprises the step of mixing the first and second compositions together. In accordance with the present invention, the mixing step can be passive or active. In one aspect of the invention, the mixing step comprises moving the combined droplet on a two-by-two sub-array of four electrodes by sequentially activating and de-activating the four electrodes to rotate the combined droplet. At least a portion of the combined droplet remains substantially stationary at or near an intersecting region of the four electrodes while the combined droplet rotates. In another aspect of the invention, the mixing step comprises sequentially activating and de-activating a linearly arranged set of electrodes of the electrode array to oscillate the combined droplet back and forth along the linearly arranged electrode set a desired number of times and at a desired frequency. Additional mixing strategies provided in accordance with the invention are described in detail herein below.

According to another embodiment of the present invention, an apparatus for manipulating droplets comprises a substrate comprising a substrate surface, an array of electrodes disposed on the substrate surface, a dielectric layer disposed on the substrate surface and covering the electrodes, and an electrode selector. The electrode selector dynamically creates a sequence of electrode pairs. Each electrode pair comprises a selected first one of the electrodes biased to a first voltage, and a selected second one of the electrodes disposed adjacent to the selected first electrode and biased to a second voltage that is less than the first voltage. Preferably, the second voltage is a ground voltage or some other reference voltage. A droplet disposed on the substrate surface moves along a desired path that runs between the electrode pairs created by the electrode selector.

According to yet another method of the present invention, a droplet is actuated by providing the droplet on a surface comprising an array of electrodes. The droplet is initially disposed on a first one of the electrodes and at least partially overlaps a second one of the electrodes that is separated from the first electrode by a first gap. The first electrode is biased to a first voltage and the second electrode is biased to a second voltage lower than the first voltage. In this manner, the droplet becomes centered on the first gap. A third one of the electrodes that is proximate to the first and second electrodes is biased to a third voltage that is higher than the second voltage to spread the droplet onto the third electrode. The bias on the first electrode is then removed to move the droplet away from the first electrode. The droplet then becomes centered on a second gap between the second and third electrodes.

According to still another method of the present invention, a droplet is split into two or more droplets. A starting droplet is provided on a surface comprising an array of electrodes. The electrode array comprises at least three electrodes comprising a first outer electrode, a medial electrode adjacent to the first outer electrode, and a second outer electrode adjacent to the medial electrode. The starting droplet is initially disposed on at least one of the three electrodes and at least partially overlaps at least one other of the three electrodes. Each of the three electrodes is biased to a first voltage to spread the initial droplet across the three electrodes. The medial electrode is biased to a second voltage lower than the first voltage to split the initial droplet into first and second split droplets. The first split droplet is formed on the first outer electrode and the second split droplet is formed on the second outer electrode.

According to a further method of the present invention, two or more droplets are merged into one droplet. First and second droplets are provided on a surface comprising an array of electrodes. The electrode array comprises at least three electrodes comprising a first outer electrode, a medial electrode adjacent to the first outer electrode, and a second outer electrode adjacent to the medial electrode. The first droplet is disposed on the first outer electrode and at least partially overlaps the medial electrode. The second droplet is disposed on the second outer electrode and at least partially overlaps the medial electrode. One of the three electrodes is selected as a destination electrode. Two or more of the three electrodes are selected for sequential biasing based on the selection of the destination electrode. The electrodes selected for sequencing are sequentially biased between a first voltage and a second voltage to move one of the first and second droplets toward the other droplet or both of the first and second droplets toward each other. The first and second droplets merge together to form a combined droplet on the destination electrode.

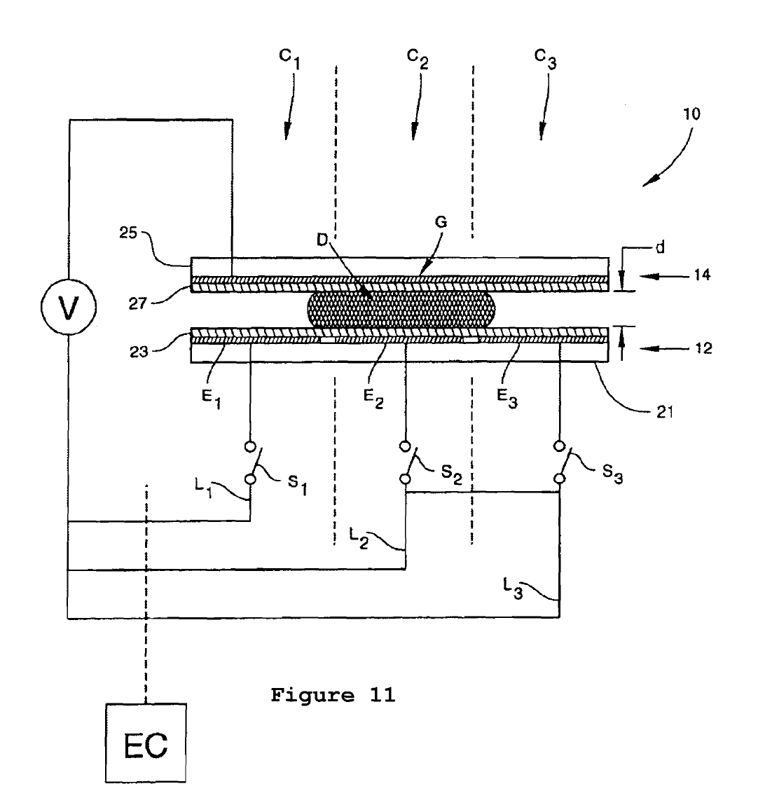

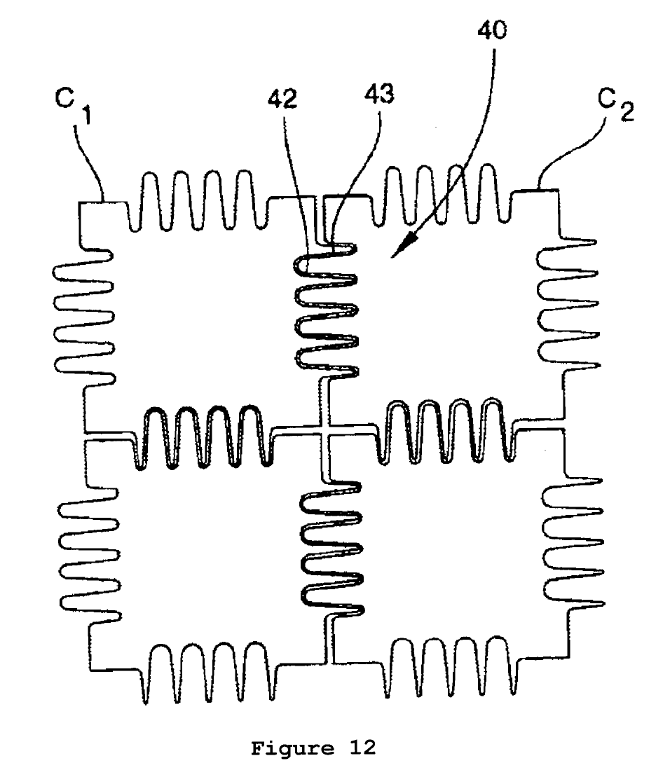

Referring now to FIG. 11, an electrowetting microactuator mechanism, generally designated 10, is illustrated as a preferred embodiment for effecting electrowetting-based manipulations on a droplet D. Droplet D is electrolytic, polarizable, or otherwise capable of conducting current or being electrically charged. Droplet D is sandwiched between a lower plane, generally designated 12, and an upper plane, generally designated 14. The terms “upper” and “lower” are used in the present context only to distinguish these two planes 12 and 14, and not as a limitation on the orientation of planes 12 and 14 with respect to the horizontal. Lower plane 12 comprises an array of independently addressable control electrodes. By way of example, a linear series of three control or drive electrodes E (specifically E.sub.1, E.sub.2, and E.sub.3) are illustrated in FIG. 11. It will be understood, however, that control electrodes E.sub.1, E.sub.2, and E.sub.3 could be arranged along a non-linear path such as a circle. Moreover, in the construction of devices benefiting from the present invention (such as a microfluidic chip), control electrodes E.sub.1, E.sub.2, and E.sub.3 will typically be part of a larger number of control electrodes that collectively form a two-dimensional electrode array or grid. FIG. 11 includes dashed lines between adjacent control electrodes E.sub.1, E.sub.2, and E.sub.3 to conceptualize unit cells, generally designated C (specifically C.sub.1, C.sub.2 and C.sub.3). Preferably, each unit cell C.sub.1, C.sub.2, and C.sub.3 contains a single control electrode, E.sub.1, E.sub.2, and E.sub.3, respectively. Typically, the size of each unit cell C or control electrode E is between approximately 0.05 mm to approximately 2 mm.

Control electrodes E.sub.1, E.sub.2, and E.sub.3 are embedded in or formed on a suitable lower substrate or plate 21. A thin lower layer 23 of hydrophobic insulation is applied to lower plate 21 to cover and thereby electrically isolate control electrodes E.sub.1, E.sub.2, and E.sub.3. Lower hydrophobic layer 23 can be a single, continuous layer or alternatively can be patterned to cover only the areas on lower plate 21 where control electrodes E.sub.1, E.sub.2 and E.sub.3 reside. Upper plane 14 comprises a single continuous ground electrode G embedded in or formed on a suitable upper substrate or plate 25. Alternatively, a plurality of ground electrodes G could be provided in parallel with the arrangement of corresponding control electrodes E.sub.1, E.sub.2 and E.sub.3, in which case one ground electrode G could be associated with one corresponding control electrode E. Preferably, a thin upper layer 27 of hydrophobic insulation is also applied to upper plate 25 to isolate ground electrode G. One non-limiting example of a hydrophobic material suitable for lower layer 23 and upper layer 27 is TEFLON® AF 1600 material (available from E. I. duPont deNemours and Company, Wilmington, Del.). The geometry of microactuator mechanism 10 and the volume of droplet D are controlled such that the footprint of droplet D overlaps at least two control electrodes (e.g., E.sub.1 and E.sub.3) adjacent to the central control electrode (e.g., E.sub.2) while also making contact with upper layer 27. Preferably, this is accomplished by specifying a gap or spacing d, which is defined between lower plane 12 and upper plane 14 as being less than the diameter that droplet D would have in an unconstrained state. Typically, the cross-sectional dimension of spacing d is between approximately 0.01 mm to approximately 1 mm. Preferably, a medium fills gap d and thus surrounds droplet D. The medium can be either an inert gas such as air or an immiscible fluid such as silicone oil to prevent evaporation of droplet D.

Ground electrode G and control electrodes E.sub.1, E.sub.2 and E.sub.3 are placed in electrical communication with at least one suitable voltage source V, which preferably is a DC voltage source but alternatively could be an AC voltage source, through conventional conductive lead lines L.sub.1, L.sub.2 and L.sub.3. Each control electrode E.sub.1, E.sub.2 and E.sub.3 is energizable independently of the other control electrodes E.sub.1, E.sub.2 and E.sub.3. This can be accomplished by providing suitable switches S.sub.1, S.sub.2 and S.sub.3 communicating with respective control electrodes E.sub.1, E.sub.2 and E.sub.3, or other suitable means for independently rendering each control electrode E.sub.1, E.sub.2 and E.sub.3 either active (ON state, high voltage, or binary 1) or inactive (OFF state, low voltage, or binary 0). In other embodiments, or in other areas of the electrode array, two or more control electrodes E can be commonly connected so as to be activated together.

The structure of electrowetting microactuator mechanism 10 can represent a portion of a microfluidic chip, on which conventional microfluidic and/or microelectronic components can also be integrated. As examples, the chip could also include resistive heating areas, microchannels, micropumps, pressure sensors, optical waveguides, and/or biosensing or chemosensing elements interfaced with MOS (metal oxide semiconductor) circuitry.

Referring now to FIG. 12, an electrode array or portion thereof is illustrated in which each structural interface between adjacent unit cells (e.g., C.sub.1 and C.sub.2) associated with control electrodes (not shown) is preferably characterized by an interdigitated region, generally designated 40, defined by interlocking projections 42 and 43 extending outwardly from the main planar structures of respective unit cells C.sub.1 and C.sub.2. Such interdigitated regions 40 can be useful in rendering the transition from one unit cell (e.g., C.sub.1) to an adjacent unit cell (e.g., C.sub.2) more continuous, as opposed to providing straight-edged boundaries at the cell-cell interfaces. It will be noted, however, that the electrodes or unit cells according to any embodiment of the invention can have any polygonal shape that is suitable for constructing a closely-packed two-dimensional array, such as a square or octagon.

Referring now to FIG. 11, the basic electrowetting technique enabled by the design of microactuator mechanism 10 will now be described. Initially, all control electrodes (i.e., control electrode E.sub.2 on which droplet D is centrally located and adjacent control electrodes E.sub.1 and E.sub.3) are grounded or floated, and the contact angle everywhere on droplet D is equal to the equilibrium contact angle associated with that droplet D. When an electrical potential is applied to control electrode E.sub.2 situated underneath droplet D, a layer of charge builds up at the interface between droplet D and energized control electrode E.sub.2, resulting in a local reduction of the interfacial energy γ.sub.SL. Since the solid insulator provided by lower hydrophobic insulating layer 23 controls the capacitance between droplet D and control electrode E.sub.2, the effect does not depend on the specific space-charge effects of the electrolytic liquid phase of droplet D, as is the case in previously developed uninsulated electrode implementations.

The voltage dependence of the interfacial energy reduction is described by:

$${γ}_{SL} ( V ) = {γ}_{SL} ( O ) - \frac{ɛ}{2 d} {V}^{2}$$

where .epsilon. is the permittivity of the insulator, d is the thickness of the insulator, and V is the applied potential. The change in γ.sub.SL acts through Young's equation to reduce the contact angle at the interface between droplet D and energized control electrode E.sub.2. If a portion of droplet D also overlaps a grounded electrode E.sub.1 or E.sub.3, the droplet meniscus is deformed asymmetrically and a pressure gradient is established between the ends of droplet D, thereby resulting in bulk flow towards the energized electrode E.sub.1 or E.sub.3. For example, droplet D can be moved to the left (i.e., to unit cell C.sub.1) by energizing control electrode E.sub.1 while maintaining control electrodes E.sub.2 and E.sub.3 at the ground state. As another example, droplet D can be moved to the right (i.e., to unit cell C.sub.3) by energizing control electrode E.sub.3 while maintaining control electrodes E.sub.1 and E.sub.2 at the ground state.

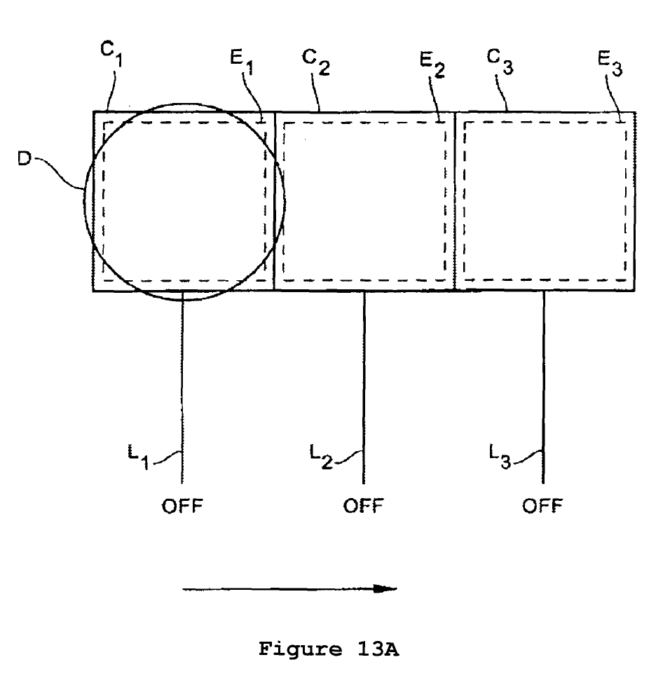

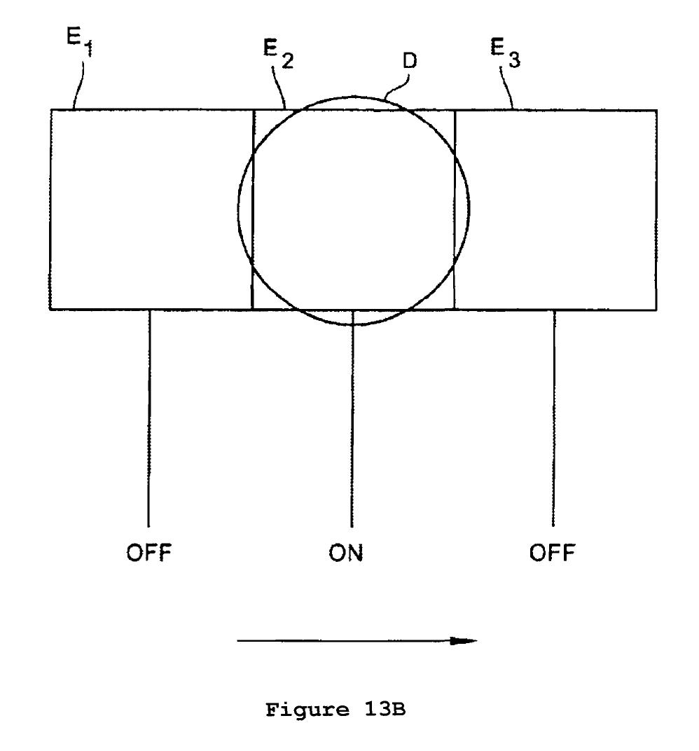

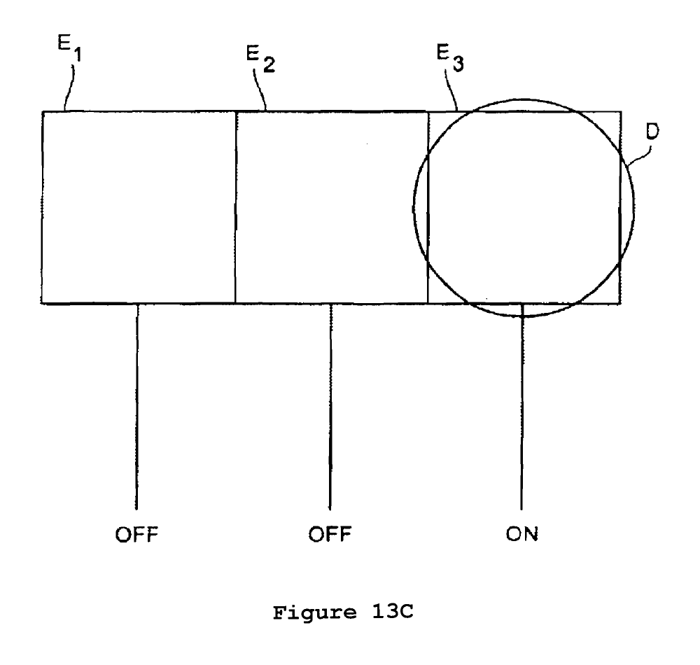

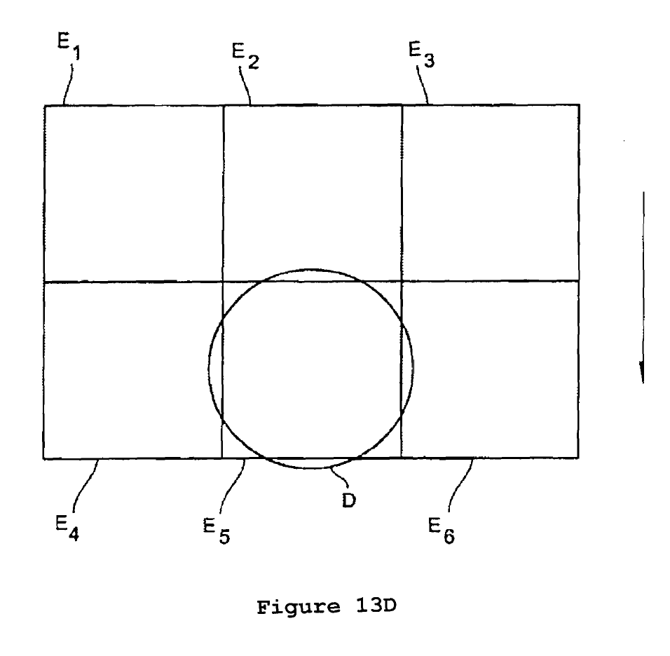

Referring now to FIGS. 13A-13B, examples of some basic droplet-manipulative operations are illustrated. As in the case of FIG. 11, a linear arrangement of three unit cells C.sub.1, C.sub.2 and C.sub.3 and associated control electrodes E.sub.1, E.sub.2 and E.sub.3 are illustrated, again with the understanding that these unit cells C.sub.1, C.sub.2 and C.sub.3 and control electrodes E.sub.1, E.sub.2 and E.sub.3 can form a section of a larger linear series, non-linear series, or two-dimensional array of unit cells/control electrodes. For convenience, in FIGS. 13A-13B, corresponding control electrodes and unit cells are collectively referred to as control electrodes E.sub.1, E.sub.2 and E.sub.3. Moreover, unit cells C.sub.1, C.sub.2, and C.sub.3 can be physical entities, such as areas on a chip surface, or conceptual elements. In each of FIGS. 13A-13B, an active (i.e., energized) control electrode E.sub.1, E.sub.2, or E.sub.3 is indicated by designating its associated electrical lead line L.sub.1, L.sub.2, or L.sub.3 “ON”, while an inactive (i.e., de-energized, floated, or grounded) control electrode E.sub.1, E.sub.2, or E.sub.3 is indicated by designating its associated electrical lead line L.sub.1, L.sub.2, or L.sub.3 “OFF”.

Turning to FIGS. 13A-13D, a basic MOVE operation is illustrated. FIG. 13A illustrates a starting position at which droplet D is centered on control electrode E.sub.1. Initially, all control electrodes E.sub.1, E.sub.2 and E.sub.3 are grounded so that droplet D is stationary and in equilibrium on control electrode E.sub.1. Alternatively, control electrode E.sub.1 could be energized while all adjacent control electrodes (e.g., E.sub.2) are grounded so as to initially maintain droplet D in a “HOLD” or “STORE” state, and thereby isolate droplet D from adjoining regions of an array where other manipulative operations might be occurring on other droplets. To move droplet D in the direction indicated by the arrow in FIGS. 13A-13B, control electrode E.sub.2 is energized to attract droplet D and thereby cause droplet D to move and become centered on control electrode E.sub.2, as shown in FIG. 13B. Subsequent activation of control electrode E.sub.3, followed by removal of the voltage potential at control electrode E.sub.2, causes droplet D to move onto control electrode E.sub.3 as shown in FIG. 13C. This sequencing of electrodes can be repeated to cause droplet D to continue to move in the desired direction indicated by the arrow. It will also be evident that the precise path through which droplet D moves across the electrode array is easily controlled by appropriately programming an electronic control unit (such as a conventional microprocessor) to activate and de-activate selected electrodes of the array according to a predetermined sequence. Thus, for example, droplet D can be actuated to make right- and left-hand turns within the array. For instance, after droplet D has been moved to control electrode E.sub.2 from E.sub.1 as shown in FIG. 13B, droplet D can then be moved onto control electrode E.sub.5 of another row of electrodes E.sub.4-E.sub.6 as shown in FIG. 13D. Moreover, droplet D can be cycled back and forth (e.g., shaken) along a desired number of unit cells and at a desired frequency for various purposes such as agitation of droplet D.

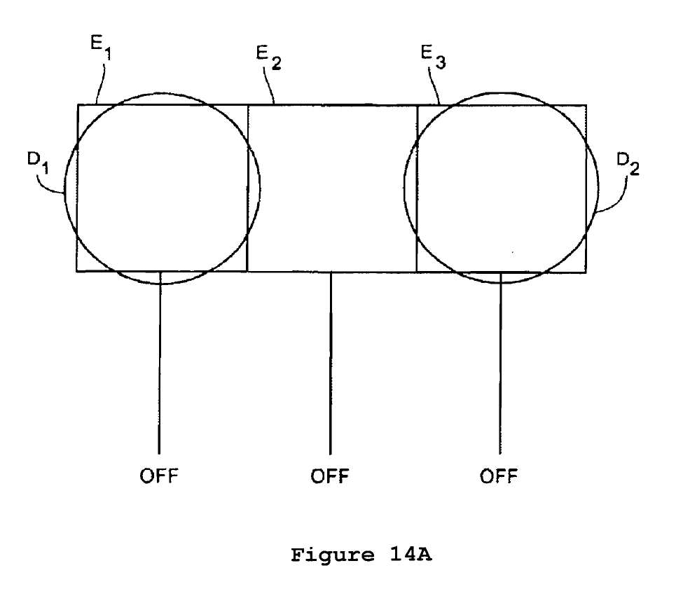

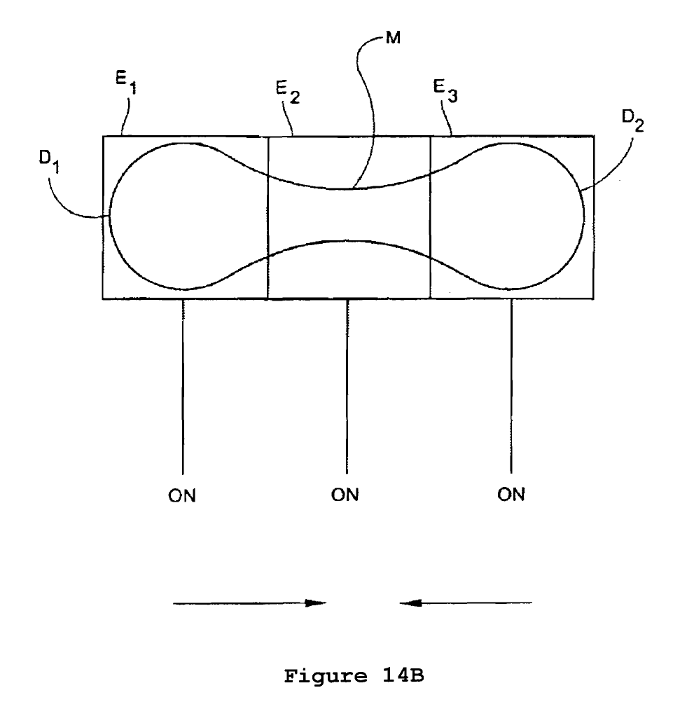

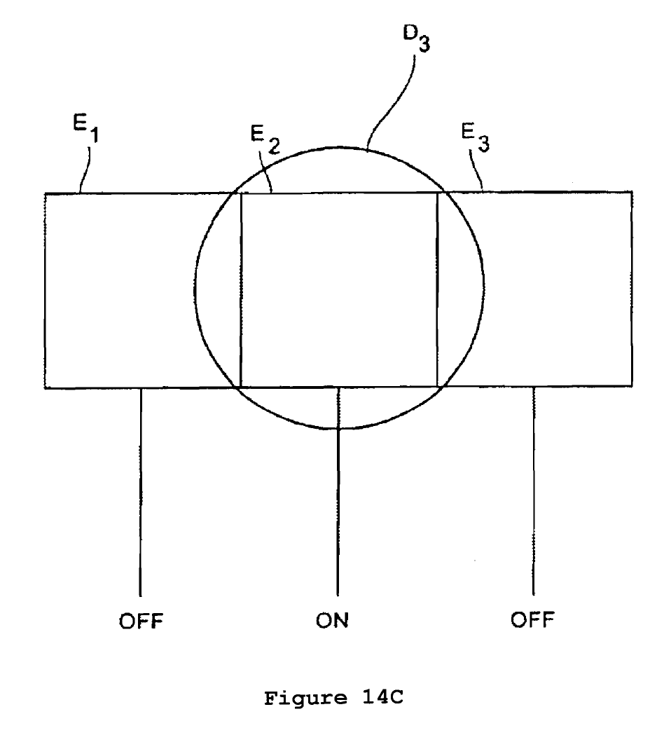

FIGS. 14A-14C illustrate a basic MERGE or MIX operation wherein two droplets D.sub.1 and D.sub.2 are combined into a single droplet D.sub.3. In FIG. 14A, two droplets D.sub.1 and D.sub.2 are initially positioned at control electrodes E.sub.1 and E.sub.3 and separated by at least one intervening control electrode E.sub.2. As shown in FIG. 14B, all three control electrodes E.sub.1, E.sub.2 and E.sub.3 are then activated, thereby drawing droplets D.sub.1 and D.sub.2 toward each other across central control electrode E.sub.2 as indicated by the arrows in FIG. 14B. Once the opposing sides of droplets D.sub.1 and D.sub.2 encounter each other at central control electrode E.sub.2, a single meniscus M is created that joins the two droplets D.sub.1 and D.sub.2 together. As shown in FIG. 14C, the two outer control electrodes E.sub.1 and E.sub.3 are then returned to the ground state, thereby increasing the hydrophobicity of the surfaces of the unit cells associated with outer electrodes E.sub.1 and E.sub.3 and repelling the merging droplets D.sub.1 and D.sub.2, whereas energized central control electrode E.sub.2 increases the wettability of its proximal surface contacting droplets D.sub.1 and D.sub.2. As a result, droplets D.sub.1 and D.sub.2 combine into a single mixed droplet D.sub.3 as shown in FIG. 14C, which represents the lowest energy state possible for droplet D.sub.3 under these conditions. The resulting combined droplet D.sub.3 can be assumed to have twice the volume or mass as either of the original, non-mixed droplets D.sub.1 and D.sub.2, since parasitic losses are negligible or zero. This is because evaporation of the droplet material is avoided due to the preferable use of a filler fluid (e.g., air or an immiscible liquid such as silicone oil) to surround the droplets, because the surfaces contacting the droplet material (e.g., upper and lower hydrophobic layers 27 and 23 shown in FIG. 11) are low-friction surfaces, and/or because the electrowetting mechanism employed by the invention is non-thermal.

In the present discussion, the terms MERGE and MIX have been used interchangeably to denote the combination of two or more droplets. This is because the merging of droplets does not in all cases directly or immediately result in the complete mixing of the components of the initially separate droplets. Whether merging results in mixing can depend on many factors. These factors can include the respective compositions or chemistries of the droplets to be mixed, physical properties of the droplets or their surroundings such as temperature and pressure, derived properties of the droplets such as viscosity and surface tension, and the amount of time during which the droplets are held in a combined state prior to being moved or split back apart. As a general matter, the mechanism by which droplets are mixed together can be categorized as either passive or active mixing. In passive mixing, the merged droplet remain on the final electrode throughout the mixing process. Passive mixing can be sufficient under conditions where an acceptable degree of diffusion within the combined droplet occurs. In active mixing, on the other hand, the merged droplet is then moved around in some manner, adding energy to the process to effect complete or more complete mixing. Active mixing strategies enabled by the present invention are described herein below.

It will be further noted that in the case where a distinct mixing operation is to occur after a merging operation, these two operations can occur at different sections or areas on the electrode array of the chip. For instance, two droplets can be merged at one section, and one or more of the basic MOVE operations can be implemented to convey the merged droplet to another section. An active mixing strategy can then be executed at this other section or while the merged droplet is in transit to the other section, as described herein below.

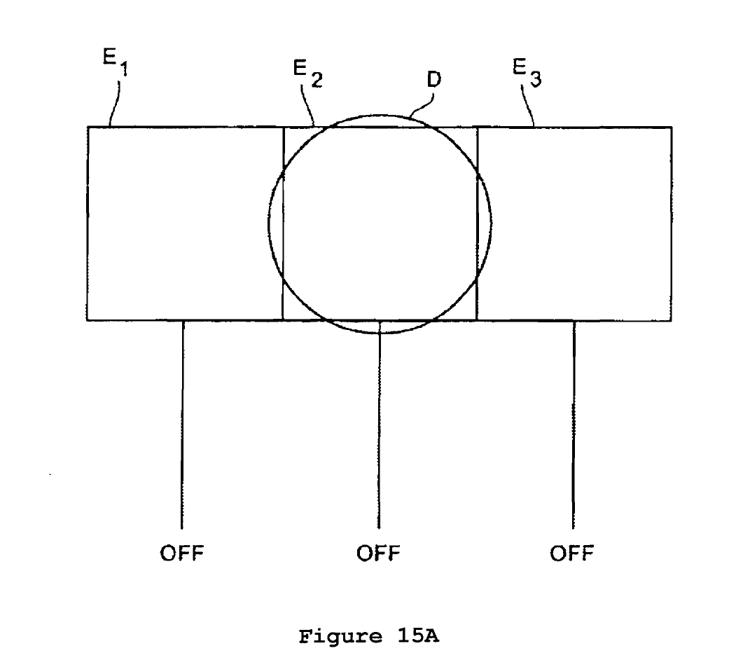

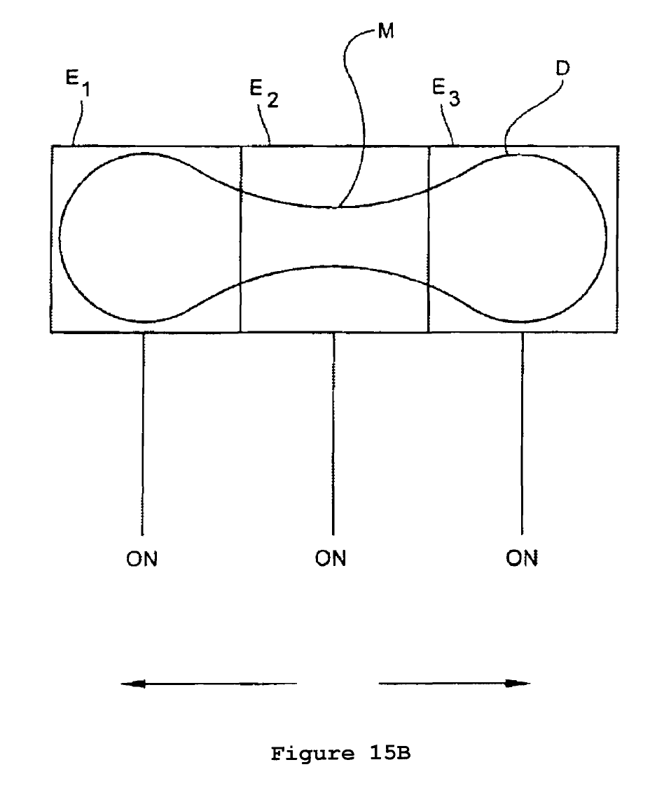

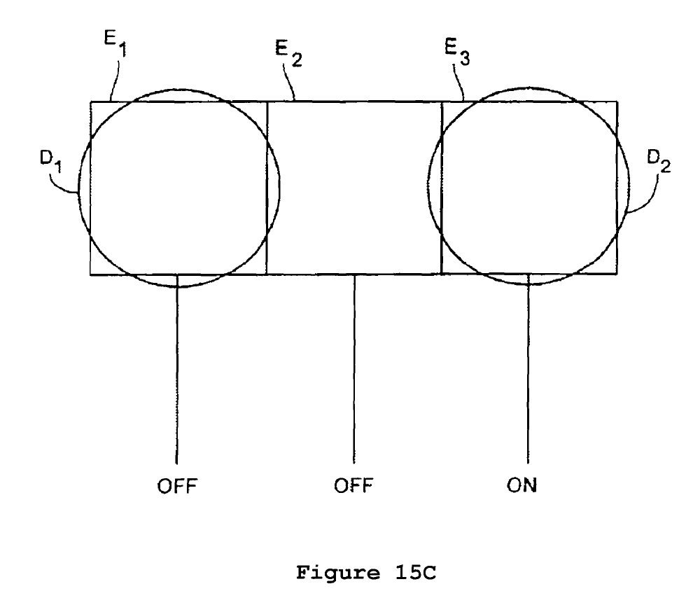

FIGS. 15A-15C illustrate a basic SPLIT operation, the mechanics of which are essentially the inverse of those of the MERGE or MIX operation just described. Initially, as shown in FIG. 15A, all three control electrodes E.sub.1, E.sub.2 and E.sub.3 are grounded, so that a single droplet D is provided on central control electrode E.sub.2 in its equilibrium state. As shown in FIG. 15B, outer control electrodes E.sub.1 and E.sub.3 are then energized to draw droplet D laterally outwardly (in the direction of the arrows) onto outer control electrodes E.sub.1 and E.sub.3. This has the effect of shrinking meniscus M of droplet D, which is characterized as “necking” with outer lobes being formed on both energized control electrodes E.sub.1 and E.sub.3. Eventually, the central portion of meniscus M breaks, thereby creating two new droplets D.sub.1 and D.sub.2 split off from the original droplet D as shown in FIG. 15C. Split droplets D.sub.1 and D.sub.2 have the same or substantially the same volume, due in part to the symmetry of the physical components and structure of electrowetting microactuator mechanism 10 (FIG. 11), as well as the equal voltage potentials applied to outer control electrodes E.sub.1 and E.sub.3. It will be noted that in many implementations of the invention, such as analytical and assaying procedures, a SPLIT operation is executed immediately after a MERGE or MIX operation so as to maintain uniformly-sized droplets on the microfluidic chip or other array-containing device.

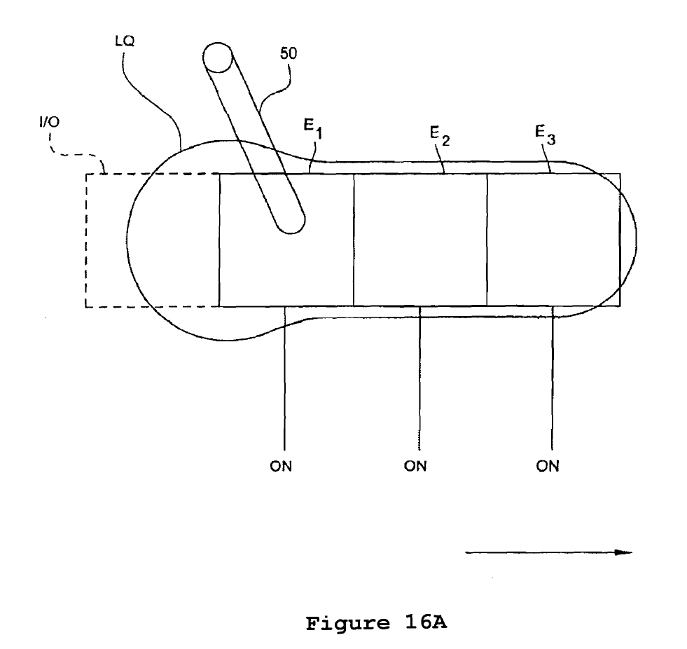

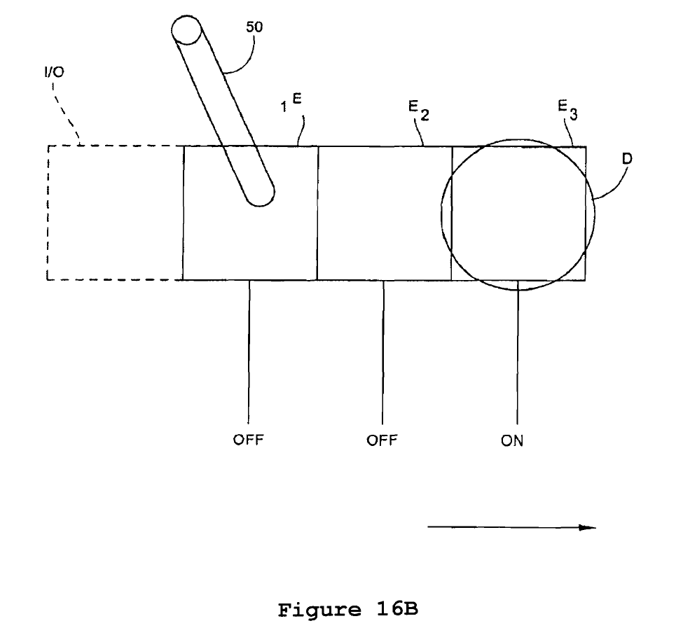

Referring now to FIGS. 16A and 16B, a DISCRETIZE operation can be derived from the basic SPLIT operation. As shown in FIG. 16A, a surface or port I/O is provided either on an electrode grid or at an edge thereof adjacent to electrode-containing unit cells (e.g., control electrode E.sub.1), and serves as an input and/or output for liquid. A liquid dispensing device 50 is provided, and can be of any conventional design (e.g., a capillary tube, pipette, fluid pen, syringe, or the like) adapted to dispense and/or aspirate a quantity of liquid LQ. Dispensing device 50 can be adapted to dispense metered doses (e.g., aliquots) of liquid LQ or to provide a continuous flow of liquid LQ, either at port I/O or directly at control electrode E.sub.1. As an alternative to using dispensing device 50, a continuous flow of liquid LQ could be conducted across the surface of a microfluidic chip, with control electrodes E.sub.1, E.sub.2, and E.sub.3 being arranged either in the direction of the continuous flow or in a non-collinear (e.g., perpendicular) direction with respect to the continuous flow. In the specific, exemplary embodiment shown in FIG. 16A, dispensing device 50 supplies liquid LQ to control electrode E.sub.1.

To create a droplet on the electrode array, the control electrode directly beneath the main body of liquid LQ (control electrode E.sub.1) and at least two control electrodes adjacent to the edge of the liquid body (e.g., control electrodes E.sub.1 and E.sub.3) are energized. This causes the dispensed body of liquid LQ to spread across control electrodes E.sub.1 and E.sub.2 as shown in FIG. 16A. In a manner analogous to the SPLIT operation described hereinabove with reference to FIGS. 15A-15C, the intermediate control electrode (control electrode E.sub.2) is then de-energized to create a hydrophobic region between two effectively hydrophilic regions. The liquid meniscus breaks above the hydrophobic region to form or “pinch off” a new droplet D, which is centered on control electrode E.sub.3 as shown in FIG. 16B. From this point, further energize/de-energize sequencing of other electrodes of the array can be effected to move droplet D in any desired row-wise and/or column-wise direction to other areas on the electrode array. Moreover, for a continuous input flow of liquid LQ, this dispensing process can be repeated to create a train of droplets on the grid or array, thereby discretizing the continuous flow. As described in more detail herein below, the discretization process is highly useful for implementing droplet-based processes on the array, especially when a plurality of concurrent operations on many droplets are contemplated.

The aspects of the invention thus far have been described in connection with the use of a droplet actuating apparatus that has a two-sided electrode configuration such as microactuator mechanism 10 illustrated in FIG. 11. That is, lower plane 12 contains control or drive electrodes E.sub.1-E.sub.3 and upper plane 14 contains ground electrode G. As regards microactuator mechanism 10, the function of upper plane 14 is to bias droplet D at the ground potential or some other reference potential. The grounding (or biasing to reference) of upper plane 14 in connection with the selective biasing of drive electrodes E.sub.1-E.sub.3 of lower plane 12 generates a potential difference that enables droplet D to be moved by the step-wise electrowetting technique described herein. However, in accordance with another embodiment of the invention, the design of the apparatus employed for two-dimensional electrowetting-based droplet manipulation can be simplified and made more flexible by eliminating the need for a grounded upper plane 14.

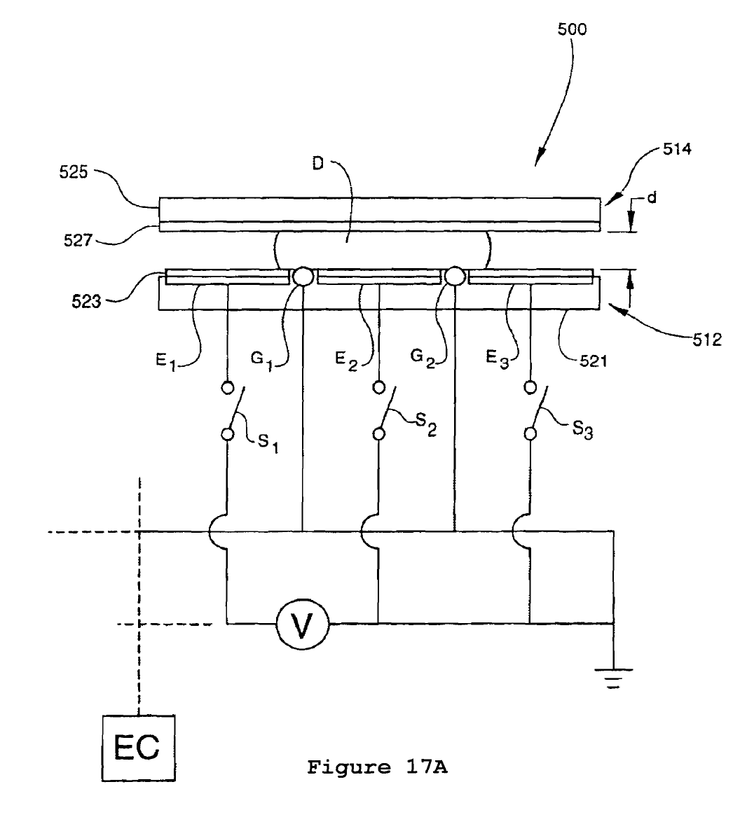

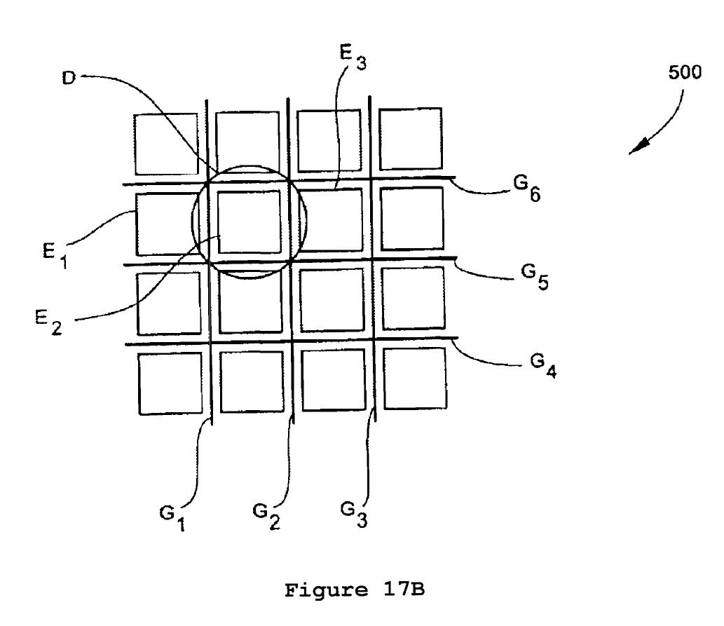

Referring now to FIGS. 17A and 17B, a single-sided electrowetting microactuator mechanism, generally designated 500, is illustrated. Microactuator mechanism 500 comprises a lower plane 512 similar to that of mechanism 10 of FIG. 11, and thus includes a suitable substrate 521 on which two-dimensional array of closely packed drive electrodes E (e.g., drive electrodes E.sub.1-E.sub.3 and others) are embedded such as by patterning a conductive layer of copper, chrome, ITO, and the like. A dielectric layer 523 covers drive electrodes E. Dielectric layer 523 is hydrophobic, and/or is treated with a hydrophobic layer (not specifically shown). As a primary difference from microactuator mechanism 10 of FIG. 11, a two-dimensional grid of conducting lines G at a reference potential (e.g., conducting lines G.sub.1-G.sub.6 and others) has been superimposed on the electrode array of microactuator mechanism 500 of FIGS. 17A and 17B, with each conducting line G running through the gaps between adjacent drive electrodes E. The reference potential can be a ground potential, a nominal potential, or some other potential that is lower than the actuation potential applied to drive electrodes E. Each conducting line G can be a wire, bar, or any other conductive structure that has a much narrower width/length aspect ratio in relation to drive electrodes E. Each conducting line G could alternatively comprise a closely packed series of smaller electrodes, but in most cases this alternative would impractical due to the increased number of electrical connections that would be required.

Importantly, the conducting line grid is coplanar or substantially coplanar with the electrode array. The conducting line grid can be embedded on lower plane 512 by means of microfabrication processes commonly used to create conductive interconnect structures on microchips. It thus can be seen that microactuator mechanism 500 can be constructed as a single-substrate device. It is preferable, however, to include an upper plane 514 comprising a plate 525 having a hydrophobic surface 527, such as a suitable plastic sheet or a hydrophobized glass plate. Unlike microactuator mechanism 10 of FIG. 11, however, upper plane 514 of microactuator mechanism 500 of FIGS. 17A and 17B does not function as an electrode to bias droplet D. Instead, upper plane 514 functions solely as a structural component to contain droplet D and any filler fluid such as an inert gas or immiscible liquid.

In the use of microactuator mechanism 500 for electrowetting-based droplet manipulations, it is still a requirement that a ground or reference connection to droplet D be maintained essentially constantly throughout the droplet transport event. Hence, the size or volume of droplet D is selected to ensure that droplet D overlaps all adjacent drive electrodes E as well as all conducting lines G surrounding the drive electrode on which droplet D resides (e.g., electrode E.sub.2 in FIG. 17B). Moreover, it is preferable that dielectric layer 523 be patterned to cover only drive electrodes E so that conducting lines G are exposed to droplet D or at least are not electrically isolated from droplet D. At the same time, however, it is preferable that conducting lines G be hydrophobic along with drive electrodes E so as not to impair movement of droplet D. Thus, in a preferred embodiment, after dielectric layer 523 is patterned, both drive electrodes E and conducting lines G are coated or otherwise treated so as render them hydrophobic. The hydrophobization of conducting lines G is not specifically shown in FIGS. 17A and 17B. It will be understood, however, that the hydrophobic layer covering conducting lines G is so thin that an electrical contact between droplet D and conducting lines G can still be maintained, due to the porosity of the hydrophobic layer.

To operate microactuator mechanism 500, a suitable voltage source V and electrical lead components are connected with conducting lines G and drive electrodes E. Because conducting lines G are disposed in the same plane as drive electrodes E, application of an electrical potential between conducting lines G and a selected one of drive electrodes E.sub.1, E.sub.2, or E.sub.3 (with the selection being represented by switches S.sub.1-S.sub.3 in FIG. 17A) establishes an electric field in the region of dielectric layer 523 beneath droplet D. Analogous to the operation of microactuator mechanism 10 of FIG. 11, the electric field in turn creates a surface tension gradient to cause droplet D overlapping the energized electrode to move toward that electrode (e.g., drive electrode E.sub.3 if movement is intended in right-hand direction in FIG. 17A). The electrode array can be sequenced in a predetermined manner according to a set of software instructions, or in real time in response to a suitable feedback circuit.

It will thus be noted that microactuator mechanism 500 with its single-sided electrode configuration can be used to implement all functions and methods described hereinabove in connection with the two-sided electrode configuration of FIG. 11, e.g., dispensing, transporting, merging, mixing, incubating, splitting, analyzing, monitoring, reacting, detecting, disposing, and so on to realize a miniaturized lab-on-a-chip system. For instance, to move droplet D shown in FIG. 17B to the right, drive electrodes E.sub.2 and E.sub.3 are activated to cause droplet D to spread onto drive electrode E.sub.3. Subsequent de-activation of drive electrode E.sub.2 causes droplet D to relax to a more favorable lower energy state, and droplet D becomes centered on drive electrode E.sub.3. As another example, to split droplet D, drive electrodes E.sub.1, E.sub.2 and E.sub.3 are activated to cause droplet D to spread onto drive electrodes E.sub.1 and E.sub.3. Drive electrode E.sub.2 is then de-activated to cause droplet D to break into two droplets respectively centered on drive electrodes E.sub.1 and E.sub.3.

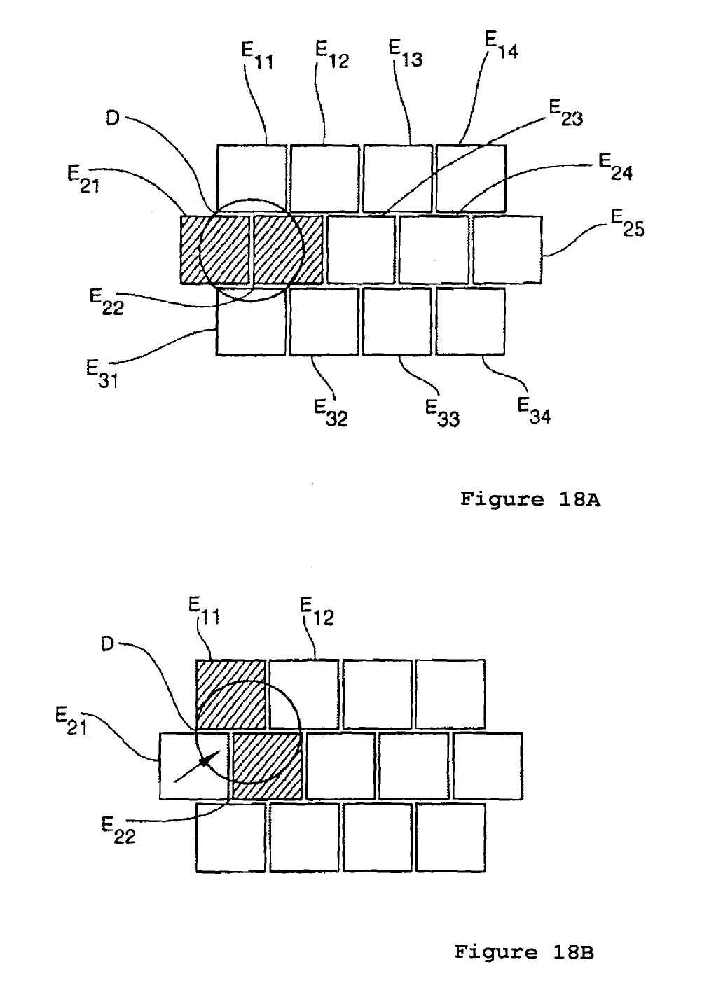

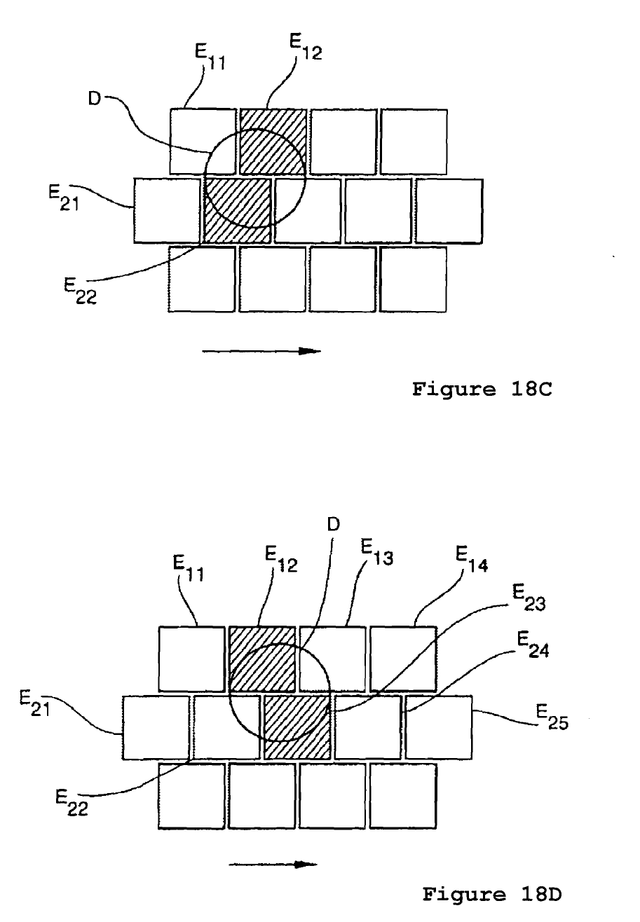

Referring now to FIGS. 18A-18D, an alternative single-sided electrode configuration is illustrated in accordance with the present invention. A base substrate containing an array of row and column biasing electrodes E.sub.ij is again utilized as in previously described embodiments. Referring specifically to FIG. 18A, an array or portion of an array is shown in which three rows of electrodes E.sub.11-E.sub.14, E.sub.21-E.sub.25, and E.sub.31-E.sub.34, respectively, are provided. The rows and columns of the electrode array can be aligned as described herein for other embodiments of the invention. Alternatively, as specifically shown in FIG. 18A, the array can be misaligned such that the electrodes in any given row are offset from the electrodes of adjacent rows. For instance, electrodes E.sub.11-E.sub.14 of the first row and electrodes E.sub.31-E.sub.34 of the third row are offset from electrodes E.sub.21-E.sub.25 of the intermediate second row. Whether aligned or misaligned, the electrode array is preferably covered with insulating and hydrophobic layers as in previously described embodiments. As in the configuration illustrated in FIGS. 17A and 17B, a top plate (not shown) can be provided for containment but does not function as an electrode.

In operation, selected biasing electrodes E.sub.ij are dynamically assigned as either driving electrodes or grounding (or reference) electrodes. To effect droplet actuation, the assignment of a given electrode as a drive electrode requires that an adjacent electrode be assigned as a ground or reference electrode to create a circuit inclusive with droplet D and thereby enable the application of an actuation voltage. In FIG. 18A, electrode E.sub.21 is energized and thus serves as the drive electrode, and electrode E.sub.22 is grounded or otherwise set to a reference potential. All other electrodes E.sub.ij of the illustrated array, or at least those electrodes surrounding the driving/reference electrode pair E.sub.21/E.sub.22, remain in an electrically floating state. As shown in FIG. 18A, this activation causes droplet D overlapping both electrodes E.sub.21 and E.sub.22 to seek an energetically favorable state by moving so as to become centered along the gap or interfacial region between electrodes E.sub.21 and E.sub.22.

In FIG. 18B, electrode E.sub.21 is deactivated and electrode E.sub.11 from an adjacent row is activated to serve as the next driving electrode. Electrode E.sub.22 remains grounded or referenced. This causes droplet D to center itself between electrodes E.sub.21 and E.sub.22 by moving in a resultant northeast direction, as indicated by the arrow. As shown in FIG. 18C, droplet D is actuated to the right along the gap between the first two electrode rows by deactivating electrode E.sub.11 and activating electrode E.sub.12. As shown in FIG. 18D, electrode E.sub.22 is disconnected from ground or reference and electrode E.sub.23 is then grounded or referenced to cause droplet D to continue to advance to the right. It can be seen that additional sequencing of electrodes E.sub.ij to render them either driving or reference electrodes can be performed to cause droplet D to move in any direction along any desired flow path on the electrode array. It can be further seen that, unlike previously described embodiments, the flow path of droplet transport occurs along the gaps between electrodes E.sub.ij as opposed to along the centers of electrodes E.sub.ij themselves. It is also observed that the required actuation voltage will in most cases be higher as compared with the configuration shown in FIGS. 17A and 17B, because the dielectric layer covers both the driving and reference electrodes and thus its thickness is effectively doubled.

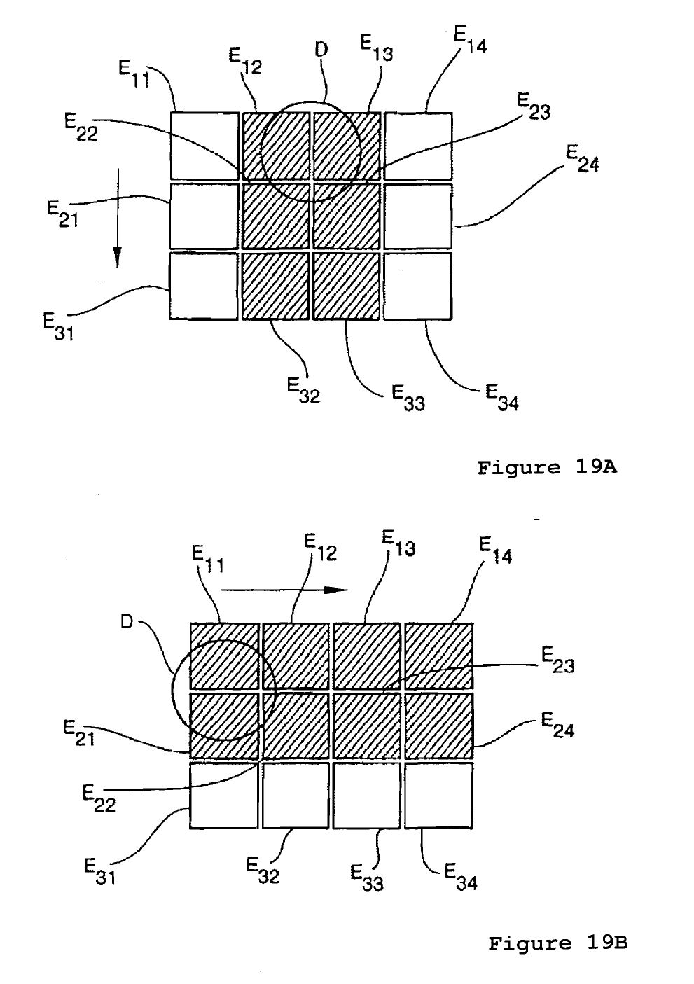

Referring now to FIGS. 19A and 19B, an electrode array with aligned rows and columns can be used to cause droplet transport in straight lines in either the north/south (FIG. 19A) or east/west (FIG. 19B) directions. The operation is analogous to that just described with reference to FIGS. 18A-18D. That is, programmable sequencing of pairs of drive and reference electrodes causes the movement of droplet D along the intended direction. In FIG. 19A, electrodes E.sub.12, E.sub.22 and E.sub.32 of one column are selectively set to a ground or reference potential and electrodes E.sub.13, E.sub.23 and E.sub.33 of an adjacent column are selectively energized. In FIG. 19B, electrodes E.sub.11, E.sub.12, E.sub.13 and E.sub.14 of one row are selectively energized and electrodes E.sub.21, E.sub.22, E.sub.23 and E.sub.24 of an adjacent row are selectively grounded or otherwise referenced.

It will be noted that a microactuator mechanism provided with the alternative single-sided electrode configurations illustrated in FIGS. 18A-18D and FIGS. 19A and 19B can be used to implement all functions and methods described hereinabove in connection with the two-sided electrode configuration of FIG. 11. For instance, to split droplet D in either of the alternative configurations, three or more adjacent electrodes are activated to spread droplet D and an appropriately selected intervening electrode is then de-activated to break droplet D into two droplets.

The present invention also provides an apparatus adapted for electrostatically actuating a droplet, and preferably an array of droplets, out from one plane to another plane (i.e., z-axis actuation). The apparatus generally comprises a first plane on which droplets are initially supplied, an elongate intermediate element spaced from the first plane, and a second plane spaced from the intermediate element that serves as the destination for actuated droplets. The first and second planes and the elongate intermediate element are rendered conductive to bring about and control the actuation of the droplet. Thus, the main structural portion of the intermediate element can be composed of a conductive (or semiconductive) material. Alternatively, the main structural portion of the intermediate element can be plated, coated, or otherwise treated with a conductive layer or film by a conventional process such as thin film deposition, plating, spin-coating, metallization, or the like. The elongate intermediate element is employed primarily to electrically ground the droplet. Thus, depending on droplet size, the axial distance between the first plane and the intermediate element is small enough to ensure that a droplet residing on the first plane contacts the intermediate element as well.

In operation, the droplet is initially placed between the first plane and the elongate intermediate element. A voltage is then applied between the intermediate element and the second plane. The droplet becomes charged and attracted to the second plane. As a result, the droplet moves from the first plane, through or around the elongate intermediate element, and into contact with the second plane. In some embodiments, the first plane is a lower plane, the second plane is an upper plane, and the intermediate element is disposed between the upper and lower plane in terms of elevation. The actuation provided by the invention is strong enough to cause the droplet to move upwards into contact with the upper plane against the opposing gravitational force. An excessively high voltage is not needed to drive the actuation because, in the microscale context in which the invention is preferably implemented, the dominant physical factor is surface tension rather than other factors such as acceleration and gravity.

In one embodiment, the intermediate element comprises an elongate element such as a wire, or a plurality of such elongate elements. Droplets move around the elongate element during actuation.

Droplets can be supplied to the apparatus by different methods. In one embodiment, the first plane includes an array of electrodes. Using an electrowetting technique, selected electrodes can be sequentially energized and de-energized to cause droplets proximate to such electrodes to move to intended positions on the array prior to being actuated. To cause movement of the droplets by electrowetting, a voltage potential is applied between the elongate intermediate element (which is typically grounded) and one or more selected electrodes of the first plane. As an alternative to electrowetting-based droplet movement, droplets can be positioned on the first plane by more conventional dosing or dispensing methods, and the first plane then moved into position underneath the intermediate element and the second plane.

According to one embodiment of the present invention, an apparatus for actuating a droplet comprises a first conductive layer, a second conductive layer, a conductive elongate medial element, and a voltage source. The first conductive layer comprises a first hydrophobic surface. The second conductive layer comprises a hydrophilic surface facing the first hydrophobic surface. The second conductive layer is axially spaced from the first conductive layer to define a gap therebetween. The conductive elongate medial element is disposed in the gap between the first and second conductive layers, and comprises a second hydrophobic surface. The voltage source communicates with the second conductive layer and the elongate medial element.

The present invention also provides a method for electrostatically actuating a droplet. A droplet is placed on a first conductive layer. The droplet is grounded by contacting the droplet with an elongate grounding element that is axially spaced from the first conductive layer. The droplet is actuated by applying a voltage potential between the grounding element and a second conductive layer axially spaced from the grounding element. The droplet becomes charged and attracted to the second conductive layer. Accordingly, the droplet moves off the first conductive layer into contact with a hydrophilic surface of the second conductive layer.

The present invention further provides a microarray structure synthesized according to the method just described. The resulting structure comprises a surface and a plurality of sample-containing spots disposed on the surface. It is therefore an object of the present invention to provide a method arid apparatus for performing non-contact electrostatic actuation of droplets from one plane to another plane. It is another object of the present invention to perform such actuation in the context of synthesizing a microarray, in which the contents of actuated droplets are stamped or printed onto a surface of the microarray.

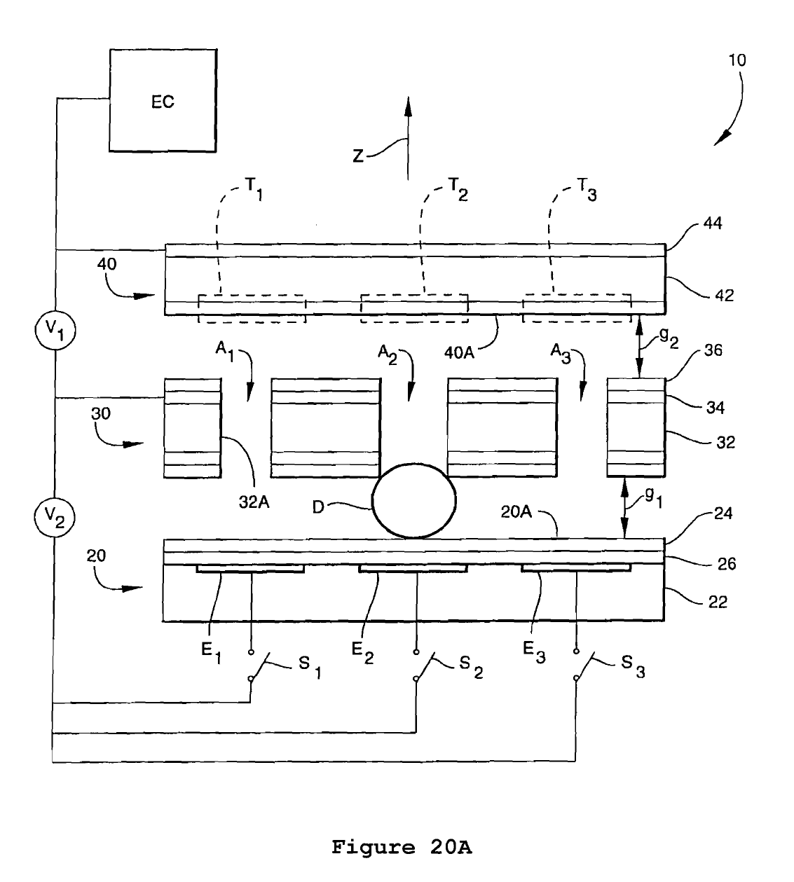

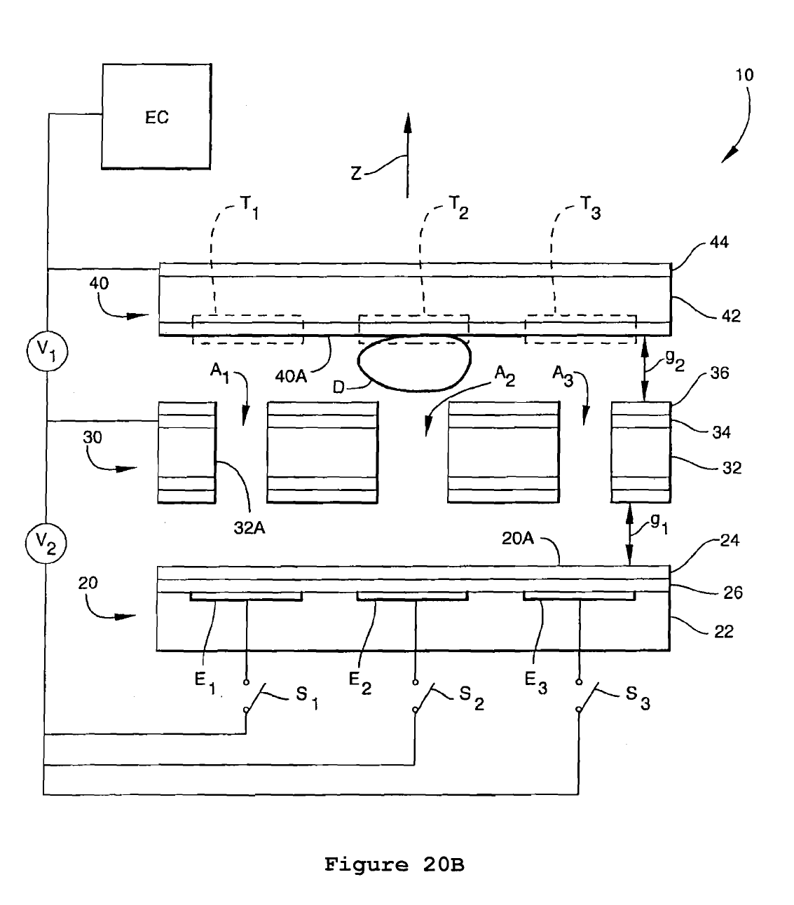

Referring now to FIG. 20A, one example of a droplet actuating apparatus, generally designated 10, has a mid-plate configuration and is illustrated for comparison with a more preferred wire traction configuration of the invention described below. Apparatus 10 comprises a first layer or plane generally designated 20, an intermediate element generally designated 30, and a second plane generally designated 40. First plane 20, intermediate element 30, and second plane 40 are generally arranged along a z-axis Z. First plane 20 is axially spaced from intermediate element 20 by a gap g.sub.1, and intermediate element 30 is axially spaced from second plane 40 by a gap g.sub.2.

First plane 20 comprises a first planar body 22 such as a plate or substrate. First planar body 22 can be composed of a non-conductive material, such as a glass or polymer, or can be a semiconductor. Typically, first planar body 22 is a dielectric material such as a glass coverslip that is rendered conductive by forming one or more control electrodes E (e.g., E.sub.1, E.sub.2, and E.sub.3 as illustrated in FIG. 20A) thereon, such as by performing a metallization process followed by a suitable masking/etching technique. A droplet-contacting surface 20A of first plane 20 is hydrophobized by providing a hydrophobic film or layer 24 on first plane. One non-limiting example of a suitable hydrophobic material is PTFE (polytetrafluoroethylene). PTFE is commercially available as the series of TEFLON® materials, such as TEFLON AF®, commercially available from E. I. duPont deNemours and Company, Wilmington, Del. Hydrophobic layer 24 also serves to electrically insulate first plane 20 (in particular, control electrodes E.sub.1, E.sub.2, and E.sub.3) from other components of apparatus 10. Alternatively, first plane 20 can be treated with a parylene coating 26 such as Parylene C coating prior to applying hydrophobic layer 24 as illustrated in FIG. 20A.

A droplet D is shown residing on control electrode E.sub.2 prior to actuation. Droplet D is electrolytic, polarizable, or otherwise capable of conducting current or being electrically charged. Typically, droplet D ranges in size between approximately 10 μm to approximately 2 mm in diameter.

In the example illustrated in FIG. 20A, intermediate element 30 is provided in the form of a medial plate 32 and is spaced along the z-axis from first plane by gap g.sub.1. The thickness of medial plate 32 is thin (e.g., 160 microns) in relation to first plane 20 and second plane 40. Medial plate 32 is perforated with an array of apertures, generally designated A (e.g., apertures A.sub.1, A.sub.2, and A.sub.3 as illustrated in FIG. 20A), having respective axes oriented substantially along the z-axis. Apertures A.sub.1, A.sub.2, and A.sub.3 can be formed by any conventional micromachining process suitable for the material used for medial plate 32, such as microdrilling, acoustic drilling, etching, and the like. To ensure that any droplet D initially provided on first plane 20 is grounded (i.e., without needing to physically actuate first plane 20 or intermediate element 30 toward each other along the z-axis), gap g.sub.1 is small enough that droplet D contacts intermediate element 30. Otherwise, droplet D might electrically float and not be actuated. Thus, gap g.sub.1 should be no greater than the size that droplet D would have in an unconstrained state.

Medial plate 32 of intermediate element 30 can be composed of a conductive material, a semi-conductive material, or a dielectric material. Preferably, medial plate 32 is a dielectric material such as a glass coverslip that is rendered conductive by applying a conductive layer 34 such as sputtered indium tin oxide (ITO). ITO is also preferred for its transparent property. During actuation as described herein below, each droplet D must move through its corresponding aperture A to reach second plane 40. Therefore, medial plate 32 is preferably hydrophobized as described above in connection with first plane 20, and thus FIG. 20A illustrates a hydrophobic layer 36 covering medial plate 32. Hydrophobic layer 36 prevents stiction between droplets D and apertures 32 during actuation, and also electrically insulates medial plate 32. Preferably, hydrophobic layer 36 also covers the inside surfaces 32A of apertures A through the thickness of medial plate 32, although for clarity this is not shown in FIG. 20A. In addition, as in the case of first plane 20, an additional insulative coating such as parylene (not specifically shown) can be provided between conductive layer 34 and hydrophobic layer 36 of medial plate 32.

Second plane 40 comprises a second planar body 42 such as a plate or substrate. Second planar body 42 can be composed of a conductive material, a semi-conductive material, or a dielectric material. Preferably, second planar body 42 comprises a derivativized glass plate. As known in the art, particularly in the field of microarray fabrication, glass plates can be derivatized by applying, for example, a poly-(L)-lysine coating. Thus, the surface of second plane 40 facing first plane, surface 40A, is hydrophilic. Second plane 40 is rendered conductive by applying a conductive layer 44 such as sputtered ITO. Second plane 40 at least conceptually comprises an array of target sites T (e.g., T.sub.1, T.sub.2, and T.sub.3 as illustrated in FIG. 20A) defined along surface 40A that designate precise locations with which actuated droplets D come into contact. In the case where control electrodes E.sub.1, E.sub.2 and E.sub.3 are associated with first plane 20, it is preferable that one target site T be aligned with one control electrode E and one aperture A. Accordingly, each droplet D provided on first plane 20 has a linear or substantially linear actuation path generally directed along the z-axis from its corresponding control electrode E of first plane 20, through its corresponding aperture A of intermediate element 30, and to its corresponding target site T of second plane 40. For purposes of multi-sample assaying and/or detection, each target site T can comprise an analyte-specific binding agent or reagent, as is commonly known in the microarray fabrication art.

FIG. 20A also schematically illustrates electrical connections made to apparatus 10. To properly effect electrostatic actuation of droplet D, a suitable voltage source V.sub.1 is connected between the respective conductive portions of second plane 40 and intermediate element 30. In the case where control electrodes E are provided with first plane 20 to control movement and positioning of droplets D, another voltage source V.sub.2 is connected between the respective conductive portions of intermediate element 30 and first plane 20. Voltage sources V.sub.1 and V.sub.2 preferably are DC voltage sources. As also shown in FIG. 20A, an electronic controller EC of suitable design (e.g., a microcontroller) is placed in communication with voltage sources V.sub.1 and V.sub.2 to control actuation and movement of droplets D. If desired, one or more of control electrodes E can be individually controlled (i.e., control electrodes E can be independently addressable). Individual control can be accomplished by providing each control electrode E or group of control electrodes E with a dedicated voltage source or, as illustrated in FIG. 20A, by providing switches S.sub.1, S.sub.2 and S.sub.3 or equivalent features.

Control electrodes E.sub.1, E.sub.2 and E.sub.3 are useful for controlling the movement and positioning of droplets D prior to or after actuation. Thus, control electrodes E.sub.1, E.sub.2 and E.sub.3 can be employed to transport one or more droplets D from another portion of the structure of apparatus 10, such as a sample reservoir or injection site, to predetermined positions on the array of first plane 20 in alignment with corresponding apertures A.sub.1, A.sub.2, and A.sub.3 of intermediate element 30 and target sites T.sub.1, T.sub.2, and T.sub.3 of second plane 40. In a case where the stamping of droplets D results in unneeded, residual liquid masses, the residual material can returned to first plane 20 and transported away from apparatus 10 by control electrodes E.sub.1, E.sub.2 and E.sub.3 across first plane 20 to an appropriate waste location. Preferably, control electrodes E.sub.1, E.sub.2 and E.sub.3 operate on the principle of electrowetting, which is described in detail by Pollack et al., “Electrowetting-based actuation of liquid droplets for microfluidic applications”, Appl. Phys. Lett., Vol. 77, p. 1725 (September 2000). Additional disclosures of electrowetting techniques and applications therefor are provided in U.S. Pat. Nos. 6,911,132 and 6,989,234, the contents of which are incorporated herein in their entirety.

Briefly, the electrowetting technique involves controlling the surface tension on droplet D, and hence the contact angles droplet D makes with the surfaces it contacts, through application of a voltage potential between intermediate element 30 (serving as the ground plane) and first plane 20. The size of droplet D and the distance of gap g.sub.1 are such that the footprint of droplet D overlaps the electrodes (e.g., control electrodes E.sub.1 and E.sub.3) adjacent to the electrode (e.g., control electrode E.sub.2), thereby allowing droplet D to be moved electrode-by-electrode to any point on the array of first plane 20. Referring to FIG. 20A and considering, as an example, the movement of droplet D from control electrode E.sub.1 to control electrode E.sub.2, both control electrodes E.sub.1 and E.sub.2 are initially grounded (i.e., switches S.sub.1 and S.sub.2 are open as illustrated), and the contact angle everywhere on droplet D is equal to the equilibrium contact angle associated with that droplet D. When an electrical potential is applied to energize control electrodes E.sub.1, and E.sub.2, a layer of charge builds up at the interface between droplet D and control electrodes E.sub.1 and E.sub.2, resulting in a local reduction of the interfacial energy γ.sub.SL. Surface 20A of first plane 20 in effect becomes hydrophilic in the vicinities of control electrodes E.sub.1 and E.sub.2, and droplet D spreads over control electrodes E.sub.1 and E.sub.2. The meniscus of droplet D is deformed asymmetrically and a pressure gradient is established between the ends of droplet D. Subsequent de-energizing of control electrode E.sub.1 renders the region of surface 20A over control electrode E.sub.1 hydrophobic once again, resulting in bulk flow of droplet D towards the remaining energized electrode E.sub.2. The voltage potential at control electrode E.sub.1 is then removed, and droplet D is centered on control electrode E.sub.2 as shown in FIG. 20A.

Referring now to FIGS. 20A and 20B, the electrostatic actuation of droplet D is now be described. As shown in FIG. 20A, droplet D is initially positioned between first plane 20 and intermediate element 30 in proper alignment with a designated aperture (e.g., A.sub.2) of intermediate element 30 and a corresponding target site (e.g., T.sub.2) of second plane 40. This positioning is accomplished either by the electrowetting technique using control electrodes E as just described, or by first loading droplet D onto first plane 20 by conventional means and moving first plane 20 with droplet D into the proper aligned position. Voltage source V.sub.1 is then used to apply a voltage between intermediate element 30 and second plane 40. Droplet D becomes charged and attracted to second plane 40. As a result, droplet D moves from first plane 20, through aperture A.sub.2, and into contact with surface 40A of second plane 40 at target site T.sub.2 as shown in FIG. 20B. Because surface 40A of second plane 40 is hydrophilic, or at least is non-hydrophobic, droplet D spreads to form a spot at its designated target site T.sub.2, as represented by the flattened shape of droplet D shown in FIG. 20B. In the case where apparatus 10 is employed for microarray stamping, an array of droplets D are actuated in this manner, resulting in the synthesis of an array of sample contents onto surface 40A of second plane 40. Second plane 40 can then be removed to interact with an appropriate analytical instrument for detection, measurement, and/or analysis of the samples of the array. Depending on the nature of target sites T, the analyte constituents of droplet D become bound to target site T by adhesion, adsorption, electrical attraction or polarization, or chemical binding, linking or reaction.

The principle underlying the droplet-based actuation process just described can be explained as follows. From Gauss' law, it is known that:

∇E=ρ/ε or,

∇²∇=−ρ/ε.

The fundamental implication of Gauss' law is that the charge density inside of any closed Gaussian surface containing a conductor can be increased by increasing the divergence of the electric field lines. An increase in surface charge density, ρ, will cause an increase in the force exerted on the surface. Hence, for a given electric field, higher forces can be achieved if the field divergence is larger. This means that if electric flux concentration is increased, a higher ρ can be obtained for the same voltage.

The example illustrated in FIGS. 20A and 20B, with its mid-plate system, requires very high voltages for actuation (e.g., 1 kV). This follows because medial plate 32 with apertures A (see FIG. 20A) shields droplet D. Thus, droplet D itself has relatively lower surface charge density, which again follows from Gauss' law. This means that the net force acting on droplet D is less than what it would be in the case where entire droplet D is exposed to the electric field. One way to increase the electric field lines converging on droplet D is to increase the applied voltage. Another way is exposing more of droplet D to the electric field. This would lead to greater charge densities on the drop. However, any aperture A cannot be larger than droplet D because droplet D would lose contact with medial plate 32 and thus would electrically float. Thus, there is a conflicting requirement. To increase the surface exposed to the field, aperture A would need to be made larger. However, aperture A cannot be so large that droplet D loses electrical contact with the medial plate 32.

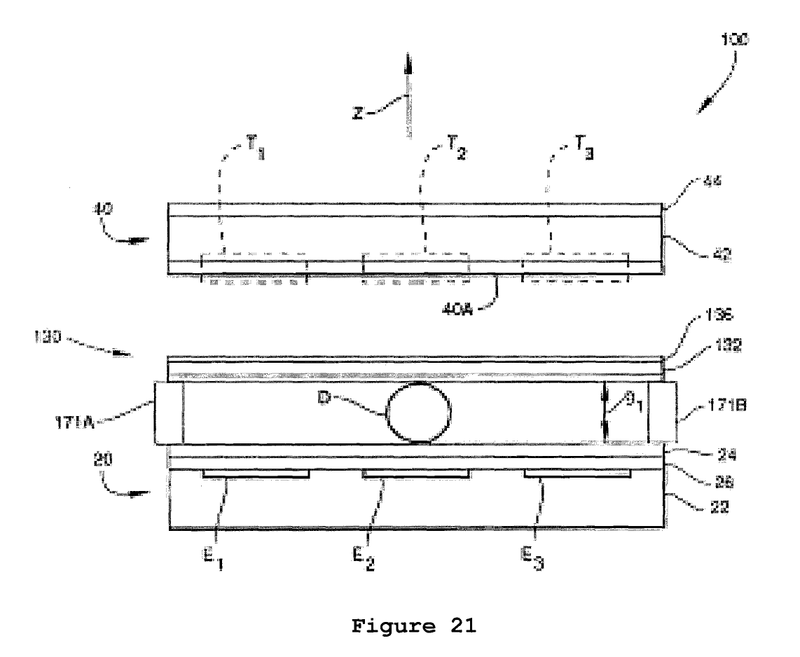

The problems attending devices such as apparatus 10 are removed with the wire traction system provided by the present invention. Referring now to FIG. 21, a droplet actuating apparatus, generally designated 100, is illustrated. Apparatus 100 is characterized by a wire traction design according to a preferred embodiment of the invention. The structure of apparatus 100 can represent a portion of a microfluidic chip, as that term is understood by persons skilled in the art, or a portion of such a microfluidic chip. The chip can be fabricated according to known technology. The chip can serve a dedicated purpose that benefits from the droplet-based electrostatic actuation disclosed herein, such as for microarray synthesis. Alternatively, apparatus 100 can be integrated with conventional microfluidic and/or microelectronic components that also are fabricated on the same chip. As examples, the chip can also include resistive heating areas or elements, microchannels, micropumps, pressure sensors, optical waveguides, and/or biosensing or chemosensing elements interfaced with MOS (metal oxide semiconductor) circuitry.

As a principal difference from apparatus 10 illustrated in FIGS. 20A and 20B, apparatus 100 provides an intermediate element 130 in the form of a conductive elongate element 132 such as a wire or narrow beam, or a parallel series of such elongate elements 132. Each elongate element 132 is strung or suspended over first plane 20 at the distance of gap g.sub.1 and supported by appropriate mounting elements such as spacer blocks 171A and 171B. The magnitude of gap g.sub.1 is selected to ensure droplet D contacts elongate element 132 when droplet D is at a pre-actuation position, and thus depends on the size or volume of droplet D. As one example, gap g.sub.1 is approximately 0.05 2 mm. Preferably, the material of elongate element 132 itself is conductive. In one successful embodiment, a platinum wire is employed. Each elongate element 132 is hydrophobized in the manner described hereinabove, as represented by hydrophobic layer 136. Elongate element 132 serves as a ground line to assist in charging droplet D and thereby effect electrostatic attraction and hence actuation. Thus, elongate element 132 is similar in function to perforated medial plate 32 (FIGS. 20A and 20B) of apparatus 10. The operation of apparatus 100 is also analogous to that of apparatus 10, except that droplet D when actuated moves around or on one side of elongate element 132 during its travel to second plane 40.

By comparison to the mid-plate configuration of apparatus 10, however, in the wire traction configuration of apparatus 100 there is a marked increase in the electric field density (and as a consequence, by Gauss' law, higher charge densities) on the surface of droplet D. The electric field strength near the surface of droplet D is nearly an order of magnitude greater than the electric field strength at a similar point in the mid-plate configuration of apparatus 10. Also, the surface area of droplet D directly under the influence of the electric field is greatly increased. The higher surface charge densities result in a higher electrostatic pressure on the droplet surface which, when integrated over the entire surface of droplet D, results in a much larger force on droplet D as compared to that in the mid-plate system illustrated in FIGS. 20A and 20B.

It will be noted that charge density could also be increased on the droplet surface by: (1) increasing the dielectric permittivity of the dielectric medium surrounding droplet D (e.g., the use of a silicone oil); (2) increasing the electrostatic drive voltage; or (3) changing the geometry of the configuration of apparatus 10 or 100 in such a way that the .gradient.sup.2.gradient. term in the equation hereinabove increases even though the magnitude of the voltage itself is constant. For apparatus 100, however, the charge density on the droplet surface has been increased without increasing the voltage. The result is a much higher charge density for the same voltage in the wire traction system of apparatus 100 as compared to the mid-plate system of apparatus 10.

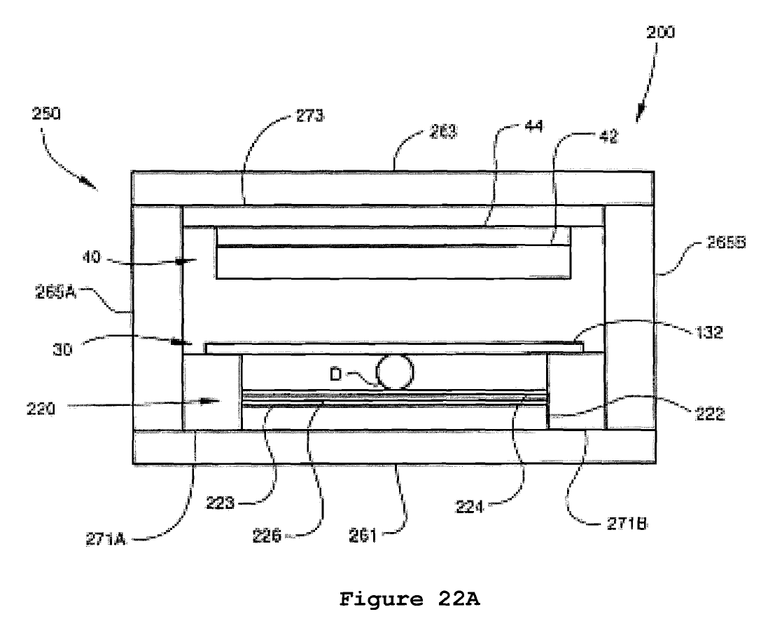

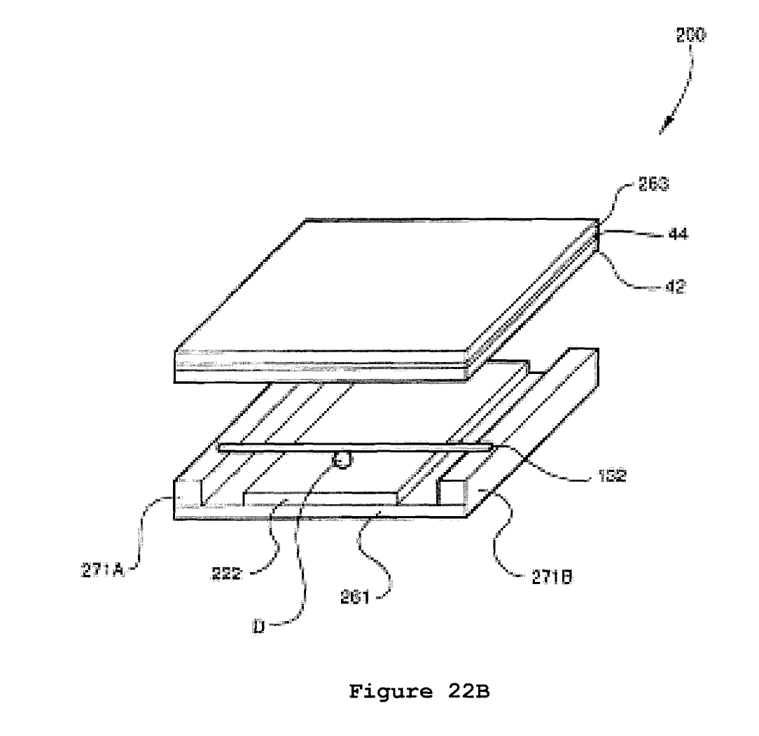

Referring now to FIGS. 22A and 22B, apparatus 100 can be encapsulated as part of a fabrication strategy for a microfluidic chip or similar device. Accordingly, FIGS. 22A and 22B illustrate an encapsulated droplet-based electrostatic actuation apparatus, generally designated 200. Apparatus 200 utilizes the wire traction system previously described with respect to apparatus 100. Thus, elongate element 132 and second plane 40 can have essentially the same design as previously described with reference to FIG. 21. FIG. 22A, however, illustrates a first plane 220 having an alternative configuration. First plane 220 is removable and does not include individual control electrodes. Instead, first plane 220 comprises a planar body 222 such as a glass slide, a continuous conductive layer 223 formed on planar body 222 such as chromium or ITO, and a hydrophobic layer 224 formed on conductive layer 223. As described hereinabove, a dielectric layer 226 such as parylene could also be disposed between conductive layer 223 and the hydrophobic layer 224. This removable design for first plane 220 could be substituted for first plane 20 employed in the embodiment described hereinabove with reference to FIG. 21.

As shown in FIG. 22A, the features comprising the wire traction system are encapsulated in a container, generally designated 250. Container 250 can take any number of forms, depending on the fabrication strategy employed to realize encapsulated apparatus 200. In the present example, container 250 is defined primarily by a lower member or base 261, an upper member or lid 263, and one or more lateral members 265A and 265B extending between lower member 261 and upper member 263 as necessary to complete the encapsulation. Lower member 261 supports first plane 220 and, preferably, one or more spacer blocks 271A and 271B that in turn support elongate member or members 132. Upper member 263 serves as a lid. Second plane 40 is attached to upper member 263 or, as illustrated, to a spacer member or adapter 273 that is in turn attached to upper member 263. First plane 220 can be loaded into container 250 prior to full encapsulation and removed after completion of an actuation process. The removability of first plane 220 is useful in a case where control electrodes or other automated means are not provided for conveying and positioning droplets D. In any case, the removability of first plane 220 facilitates replacement of first plane 220 if its hydrophobic surface 224 becomes degraded through extensive use. In addition, the internal volume of apparatus 200 encapsulated by container 250 can be flooded with a filler fluid that is immiscible with droplet D, such as silicone oil.

### EWOD Array Chip

An array-based print-head can be used to eliminate bottlenecks in scaffold fabrication by allowing for high-density rapid array printing, in seconds or minutes, rather than hours, and low and at high densities (e.g., 10⁴ spots/cm², or up to 10⁵-10⁶ spots/cm² assuming spot sizes of 7-18 μm). Theoretically, array densities achievable with picoliter droplet printing are in the range of 1×10⁵/cm² can be greater of 5×10⁵/cm². Nanoliter droplet transfer rates have been performed in the range of about 1000 transfers/sec at linear velocities in the range of about 10 cm/sec. At such rates one could populate a 700×700 array, 10 columns at a time, followed by vertical printing, all in about 1 minute. Optionally, no intermediate cleaning or preprinting steps are required. These rates represent significantly greater throughput than any existing automated arraying system.

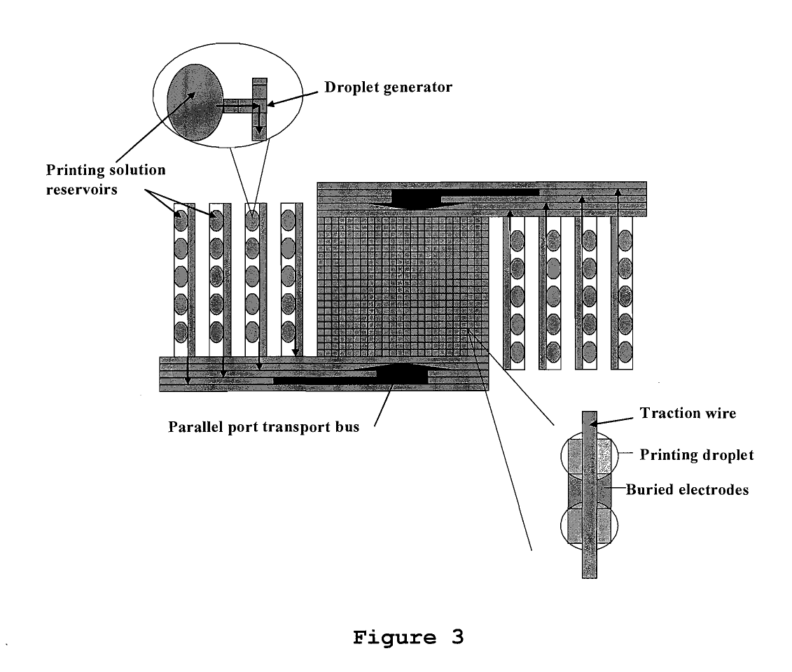

The EWOD printing system of the invention is based on self-aligned, non-contact, nanoliter or sub-nanoliter array printing using droplet-based microfluidics. One embodiment of the microfluidic printing array system of the invention is depicted in FIG. 3. The microfluidic chip has microliter reservoir pads appropriately treated to hold a variety of different solutions on the left and right sides of the chip. In some embodiments, these solutions can be loaded onto the microliter reservoir pads by robotic means. Then, from the reservoirs, nanoliter or sub-nanoliter solution droplet is automatically formed and transported on parallel buses to an electrode array. Subsequently, a desired number of droplets are transported rapidly onto the array electrodes. Once the droplets are positioned on the electrode array, wire traction electrodes above the insulator surface of the electrode array electrically ground the droplets. Upon the application of a modest voltage to a bottom substrate, all of the droplets in the array are actuated vertically to print in a self-aligned manner onto the corresponding receiving sites on the substrate. The plate can be hydrophilic to better allow the droplet to adhere to the plate. Once the droplet printing is completed, the array can be moved from the printing system.

Nanoliter-sized droplets have been dispensed using EWOD chips and the size of these droplets was on the order of 100 μm (Kolar and Fair, 2001, SmallTalk, pp. 139). Droplets (about 50-200/minute) can be delivered at a single location, so the anticipated build rate can be around 0.5-2 cm/minute for a nanoliter-volume droplet. The total size of the structure that can be built will depend on the size of microfluidic array. For picoliter-volume droplets, diameter is on the order of 10 μm and the build rate is approximately 0.05-0.2 mm/minute.

In one embodiment, the invention is an apparatus for engineering a tissue by actuating droplets by electrowetting. In some embodiments the apparatus has at least two microarray print heads, but in other embodiments can have at least three, or four, or more microarray print heads. In other embodiments, the apparatus has at least four microarray print heads. Each of the print heads of the apparatus contains a first conductive layer having an array of control electrodes covered by a first hydrophobic insulator surface. Each of the print heads of the apparatus also contains a second conductive layer having a second conductive layer surface that faces the first hydrophobic surface. The second conductive layer is spaced from the first conductive layer to define a gap between them. The second conductive layer has an actuation voltage ranging from about 10 to about 100 volts or more. Each of the print heads of the apparatus also has a wire traction system having at least one conductive elongate wire element disposed in the gap between the first and second conductive layers, and a second hydrophobic surface, having a voltage that is less than the second conductive layer actuation voltage. Each of the print heads of the apparatus has a voltage source communicating with the second conductive layer, and with the elongate wire element, that provides an actuation voltage to the second conductive layer ranging from about 10 to about 100 volts or more. Upon application of the actuation voltage, a droplet is caused to move along a pathway extending around the conductive elongate wire element and from the first conductive layer towards the second conductive layer. In some embodiments having at least four microarray print heads, one of the print heads can be used to actuate droplets of a hydrogel, one of the print heads can be used to actuate droplets of a crosslinker, one of the print heads can be used to actuate droplets of a cell suspension, and one of the print heads can be used to actuate droplets of a bioactive material, such as, for example, a growth factor.

In another embodiment, each of the print heads of the apparatus contains a substrate having a substrate surface and an array of drive electrodes disposed on the substrate surface. Each of the print heads of the apparatus also contains a dedicated array of reference elements settable to a common reference potential and disposed in at least substantially co-planar relation to the electrode array, so that the array of reference elements is electrically and physically distinct from the drive electrode array, and so that each drive electrode is adjacent to at least one of the reference elements. Each of the print heads of the apparatus also contains a dielectric layer disposed on the substrate surface to cover the drive electrodes. Each of the print heads of the apparatus also contains an electrode selector for sequentially activating and de-activating one or more selected drive electrodes of the array to sequentially bias the selected drive electrodes to an actuation voltage, so that a droplet disposed on the substrate surface moves along a desired path defined by the selected drive electrodes. In some embodiments having at least four microarray print heads, one of the print heads can be used to actuate droplets of a hydrogel, one of the print heads can be used to actuate droplets of a crosslinker, one of the print heads can be used to actuate droplets of a cell suspension, and one of the print heads can be used to actuate droplets of a bioactive material, such as, for example, a growth factor.

In another embodiment, each of the print heads of the apparatus contains a substrate having a substrate surface and an array of electrodes disposed in at least substantially co-planar relation on the substrate surface, wherein the array of electrodes contains drive electrodes and dedicated reference electrodes. Each of the print heads of the apparatus also contains a dielectric layer disposed on the substrate surface and covering the array of electrodes. Each of the print heads of the apparatus contains an electrode selector for dynamically creating a sequence of electrode pairs, each electrode pair comprising a selected one of the drive electrodes biased to a first voltage and a selected one of the reference electrodes disposed adjacent to the selected drive electrode and biased to a second voltage less than the first voltage, so that a droplet disposed on the substrate surface moves along a desired path running between the electrode pairs created by the electrode selector, and so that manipulation of the droplet is accomplished by electrowetting actuation so that the droplet overlaps a selected one of the drive electrodes and a selected one of the reference electrodes continuously. In some embodiments having at least four microarray print heads, one of the print heads can be used to actuate droplets of a hydrogel, one of the print heads can be used to actuate droplets of a crosslinker, one of the print heads can be used to actuate droplets of a cell suspension, and one of the print heads can be used to actuate droplets of a bioactive material, such as, for example, a growth factor.

In some embodiments, the invention is a method of tissue engineering by actuating droplets by electrowetting. In one embodiment, the steps of the method include the horizontal actuation of each droplet of a group of hydrogel droplets, to position the hydrogel droplets at discrete locations on a microfluidic chip, followed by the vertical actuation of the group of hydrogel droplets so that the group of hydrogel droplets is deposited on a tissue growth surface. In another embodiment, the steps of the method include the horizontal actuation of each droplet of a group of crosslinker droplets, to position the crosslinker droplets at discrete locations on a microfluidic chip, followed by the vertical actuation of the group of crosslinker droplets so that the group of crosslinker droplets is deposited on a tissue growth surface. In another embodiment, the steps of the method include the horizontal actuation of each droplet of a group of cell suspension droplets, to position the cell suspension droplets at discrete locations on a microfluidic chip, followed by the vertical actuation of the group of cell suspension droplets so that the group of cell suspension droplets is deposited on a tissue growth surface. In another embodiment, the steps of the method include the horizontal actuation of each droplet of a group of growth factor droplets, to position the growth factor droplets at discrete locations on a microfluidic chip, followed by the vertical actuation of the group of growth factor droplets so that the group of growth factor droplets is deposited on a tissue growth surface. In various embodiments, the steps of horizontally and then vertically actuating droplets of hydrogel, crosslinker, cell suspension, and growth factor are repeated to deposit a second layer, or a third layer, or a fourth layer, or additional layers up to a hundred, or up to a thousand, or up to a million or more layers of droplets onto the tissue growth surface.

### Representation of Heterogeneous Tissue Construct

In the EWOD multi-microarray printing system of the invention, at least four different kinds of materials can be combined to make soft tissue constructs: hydrogels, crosslinkers, cells and bioactive materials, such as growth factors. This multiple material scaffold can be treated as a heterogeneous object in the modeling process.

To represent a heterogeneous tissue construct, tissue geometry, as well as material distributions of the object, can be designed. The CAD model can thus be enriched with useful material information. The continuously varying material composition produces gradation in material properties, sometimes referred to as functionally gradient materials (FGM) (see Cheng and Lin, 2005, Computer-Aided Design 37:1115-1126). In the scheme of heterogeneous tissue construct modeling, in some embodiments, voxel-based volumetric datasets to represent the material variations can be used.

The core issue of heterogeneous tissue representation is to design a scheme to represent the geometry and material information in order to integrate with the CAD system and material design. First, a geometric space, S, having the macroscopic shape of the tissue, is defined. A heterogeneous solid, H, on geometric space S, is defined, such that H consists of different kinds of materials having different material composition functions within the interior, specified by the material designer. For material composition, the material space as a vector space, and the components as the material primitives, are designed. A FGM of the heterogeneous class can thus be represented; and a distance field is referred to as a distance map of all inner points to the selected features. The FGM takes the distance from inner object point to the selected feature(s) as arguments, and it must satisfy the requirement of 0<FGM<1 in the material gradient range. In various embodiments, the user interface for this EWOD microarray system includes various support modules: a voxelization module, a material evaluation module (material designer), and a 2D slicing module.

### Design of an Automatic and Intelligent Soft Tissue Manufacturing System

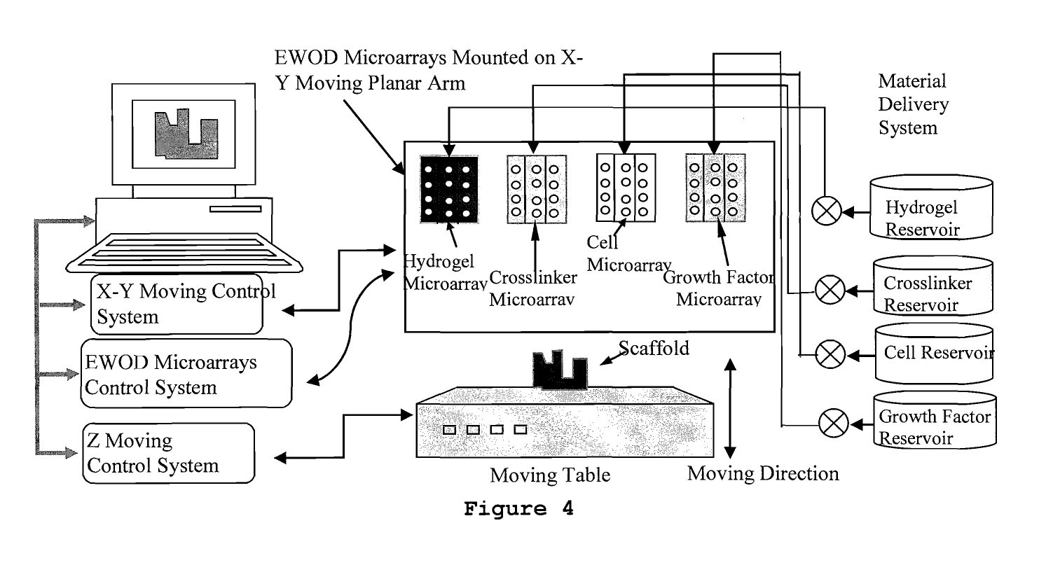

The hydrogel printing system of the invention, for freeform fabrication of tissue constructs, can deposit living cells and other biological and bioactive compounds in a biological friendly environment, for example, at temperatures ranging from about 4° C. to about 37° C. A key component of the inventive system is a multi-microarray (e.g., 2 array units, 3 array units, 4 array units, 5 array units, 6 array units, or more) printing-head that is designed to make accurate micro-deposits of various viscosity fluids, suspensions and solutions, with exceptional control (FIG. 4). The data processing system can process the scaffold models from a computer design, or from a CT or an MRI image or the like, and can convert it into a layered process tool-path. The motion control system is driven by the layered manufacturing technique. In some embodiments of the invention, the material delivery system consists of multiple microarrays with different sizes, thus enabling the deposition of specified hydrogels, and various bioactive suspension and solutions with different viscosities for constructing 3D tissue scaffolds.

The EWOD-based print head is fixed on the moving planar arm and moves in X and Y directions (i.e., horizontally) based on the scaffold CAD model (FIG. 4). In one embodiment, four reservoirs connect to four print-jet arrays to provide a variety of materials (e.g., scaffold, crosslinker, cells, bioactive materials, growth factors, etc.). The moving planar arm block contains four microarrays forming the print-head that can work sequentially in the system controlled by a CAD model based on the heterogeneous tissue construct design. The first microarray print head dispenses the scaffold material (e.g., hydrogel, chitosan solution, etc.), and then the second microarray print head dispenses the crosslinker solution (e.g, covalent, ionic, genipin, etc.) to crosslink the deposited hydrogel solution on the substrate. The third microarray print head is designed to dispense bioactive materials, suspended in solution (e.g., water, saline, serum, growth factors, culture medium). The fourth microarray print head is used to dispense living cells (e.g., cardiomyoblasts, endothelial cells, etc.) suspended in a solution (e.g., culture medium).

Under the control of a computer, the moving table moves up one layer height in the Z direction (i.e., vertically) after finishing each layer. In this manner, the hydrogel scaffold is stacked up layer by layer and a three-dimensional hydrogel scaffold can be fabricated. The multiple microarray system allows for the near-in-time deposition of cells, growth factors, and scaffold materials, thus enabling the fabrication of heterogeneous tissue scaffolds that contain living cells and bioactive compounds.

### Support Structure

Generally, SFF systems provide some kind of a support structure build mechanism to deal with overhang features on the built part. Although not specifically excluded from the system of the invention, the tissue fabrication system of the invention need not include this support structure build mechanism for at least three reasons. First, most scaffolds have self-supported internal tissue matrix and can be oriented to eliminate support structures. Second, generally, the deposited hydrogel is sticky, and thus easily sustained and interconnected. Third, hydrogel and cell densities are similar to water, and a buffer solution may be used to support the weight of the deposited hydrogel and cells. Optional elimination of the support structure can serve to simplify the system and reduce build time.

### Bio-Mimetic CAD Modeling and Reverse Engineering

Generally, the creation of a 3D CAD model is the first step in SFF. The 3D CAD model can be created directly using CAD software (commercially available examples include, Pro/E, Ideas, Solidworks, or AutoCAD) or can be created using reverse engineering, which can reconstruct the 3D model from surface coordinates or multi-planar images of cross-sections of the model. The 3D model of the imaged parts is reconstructed from these high-resolution multi-planar images through biomimetic model software. Several commercially available biomimetic software packages, such as Mimics (Materialize) and AMIRA (TGS), can be used to reconstruct a 3D model from CT and MRI images and generate an STL file, which is a commonly used data format in SFF. By way of a non-limiting example, these processes can be used to create a CAD-based flow and manufacturing model of a child's airway based on MR volume imaging. By way of a further non-limiting example, these processes can be used to create a CAD-based flow and manufacturing model of a trabecular bone model from MicroCT for SFF manufacturing of bone tissue scaffold. By way of further non-limiting example, these processes can be used to create a CAD-based flow and manufacturing model of a biomimetic myocardial model from imaging studies of rat hearts. By way of further example, myocardial ultrastructure can be mapped using serial sectioning, staining with hematoxilin and eosin, imaging by brightfield microscopy, and 3D reconstruction in AMIRA.

### Definitions:

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “layer” is used herein to denote a structure or body that is typically but not necessarily planar or substantially planar, and is typically deposited on, formed on, coats, treats, or is otherwise disposed on another structure.

The term “communicate” (e.g., a first component “communicates with” or “is in communication with” a second component) is used herein to indicate a structural, functional, mechanical, electrical, optical, or fluidic relationship, or any combination thereof, between two or more components or elements. As such, the fact that one component is said to communicate with a second component is not intended to exclude the possibility that additional components may be present between, and/or operatively associated or engaged with, the first and second components.

For purposes of the present disclosure, it is understood that when a given component such as a layer, region or substrate is referred to herein as being disposed or formed “on”, “in”, or “at” another component, that given component can be directly on the other component or, alternatively, intervening components (for example, one or more buffer layers, interlayers, electrodes or contacts) can also be present. It will be further understood that the terms “disposed on” and “formed on” are used interchangeably to describe how a given component is positioned or situated in relation to another component. Hence, the terms “disposed on” and “formed on” are not intended to introduce any limitations relating to particular methods of material transport, deposition, or fabrication.

For purposes of the present disclosure, it will be understood that when a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Example 1

### Hydrogel Dispensing

The capability of EWOD to dispense hydrogels has been assessed as follows. Several solutions were mixed, filtered, and then used as described below: a 1% w/v sodium alginate, a 2% w/v sodium alginate with a viscosity of 250 cP at 25° C., and a 1% w/v calcium chloride solution. The experiments described herein were performed on a glass chip with patterned chrome electrodes, having a pitch of 0.75 mm and a gasket to maintain top-plate height above the electrodes. The chip was first coated with Parylene C, which functions as a dielectric and chemical insulator, and then was coated with a thin layer of Teflon AF for hydrophobicity. The top plate consisted of a sputtered indium-tin oxide (ITO)-coated film, which was later coated with Teflon AF for hydrophobicity. ITO is a transparent conductor, allowing the top plate to remain grounded during operation. All of the experiments were performed at room temperature.

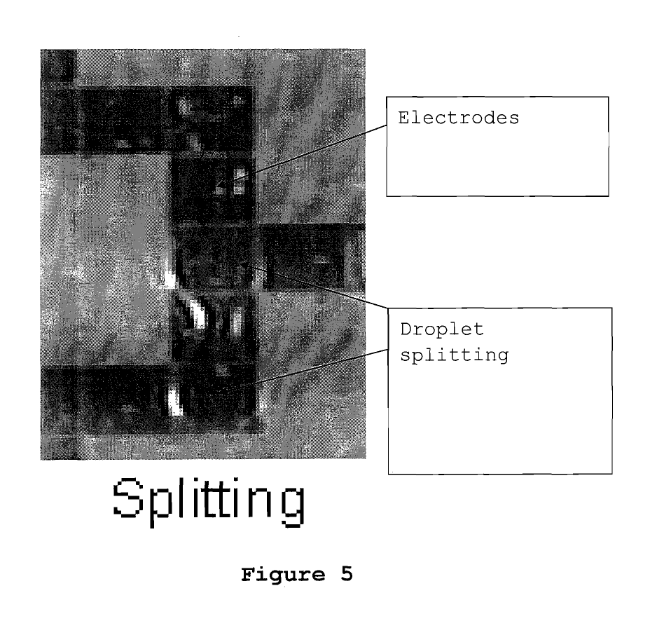

First, experiments were conducted to determine whether the alginate and crosslinking solutions could be actuated, dispensed, combined, and split. For each test, the glass chip and top plate were washed and dried, then the top plate was aligned and placed on top of the glass chip and the space between them was filled with 2 cSt silicone oil, which serves to prevent evaporation and reduce the actuation voltage. The first test was performed by filling a reservoir with 650 nl of 1% alginate. A 55V actuation voltage was chosen, ensuring a reasonable velocity without causing dielectric breakdown. The solution was dispensed by extending a ‘finger’ of the solution from the reservoir through electrowetting, then by applying voltage only to the last electrode in the ‘finger’ and the reservoir, causing the finger to narrow and eventually split between the two electrodes (FIG. 5). This solution appeared to require a longer ‘finger’ before splitting than other solutions due to the higher viscosity. The first droplet was then moved away via actuation and a second droplet was dispensed. The two droplets were combined by actuating them into each other and then split using a method similar to dispensing. Similar experiments were successfully performed with the 2% alginate solution (using a higher actuation voltage and slower velocity due to its higher viscosity of about 250 cp)), as well as with the 1% crosslinking solution (at 55V actuation voltage).

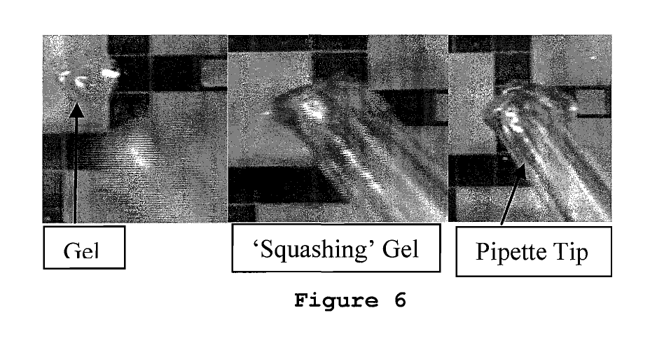

Second, experiments were conducted to determine whether the alginate solution and a crosslinking solution could be dispensed, actuated, and then combined to create the hydrogel on chip. Approximately 650 nl of the 1% alginate and the 1% Genipin crosslinking solution were inserted into their own adjacent reservoirs. The crosslinker was dispensed first, moved to a holding electrode, and then the alginate was dispensed and moved toward the crosslinker. When the droplets moved together, they rapidly became unable to be actuated, indicating a change in the surface tension or the viscosity or both the surface tension and the viscosity. To determine whether the alginate had gelled, the top plate was removed and the droplet was ‘squashed’ using a pipette tip (FIG. 6). This squashing resulted in apparently gel-like behavior. Finally, a glass slide (hydrophilic) was placed over the gel, which transferred the gel to the glass slide.

### Example 2

### Cell Manipulation on EWOD Chip

The capability of the EWOD process for handling, dispensing and actuating cell suspensions was examined. Further, the voltage that can be applied to cells without damaging them was examined.

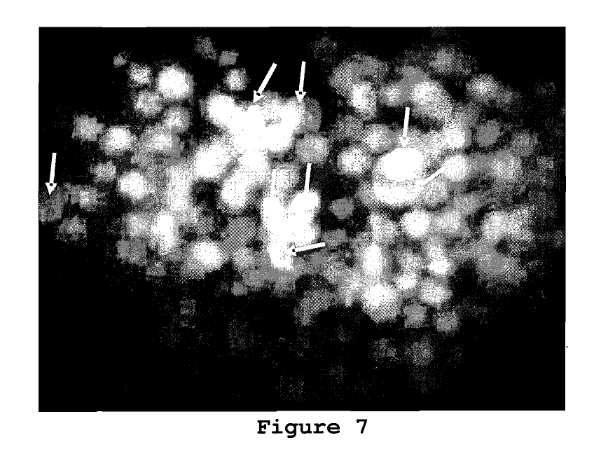

Tests were conducted on the EWOD chip using the human fetal osteoblast cell line hFOBs 1.19 (obtained from ATCC between passage 11 and 13). Cells were cultured in Dulbecco's Modified Eagles Medium (DMEM) containing 10% FBS and 1% Penicillin-Streptomycin prior to the experiment. Cultured cells were trypsinised, suspended in PBS and separated by centrifuging. The separated cells were treated with a Live Dead Assay (Molecular Probes) reagent solution (6 μM ethidium homodimer-1 and 2 μM Calcein in PBS) according to the manufacturer's instructions. The cell suspension was loaded on chips, which were actuated with voltages ranging from 40-60 V. After actuation the EWOD chips were observed under fluorescent microscope to quantify live and dead cells. The fraction of live cells after actuation was about 94% (FIG. 7), which was comparable to the fraction of live cells in the solution before they were loaded onto the chip. Even after two hours with 60V applied to the switching electrodes, there was no noticeable change in the ratio of live-to-dead cells in the immersed droplet. Overall, the experiments demonstrated that the proposed method of EWOD is feasible for dispensing living cells.

### Example 3

### EWOD-Based Vertical Actuation of Droplets Ranging in Size Between Nanoliter-Sized and Microliter-Sized

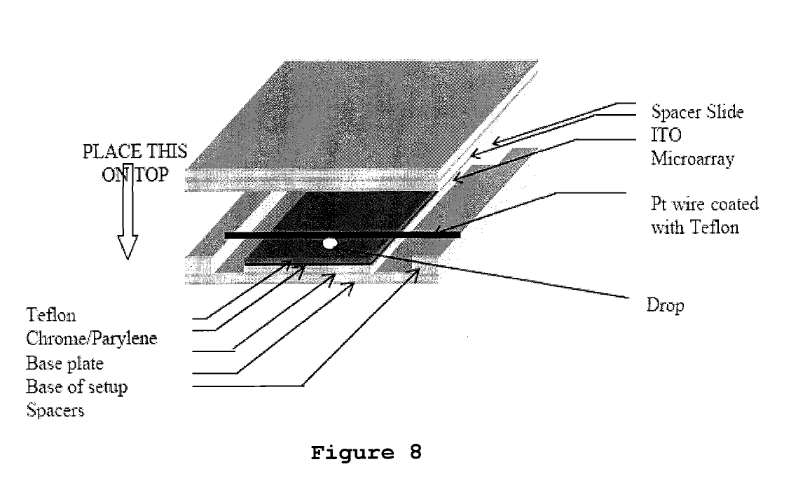

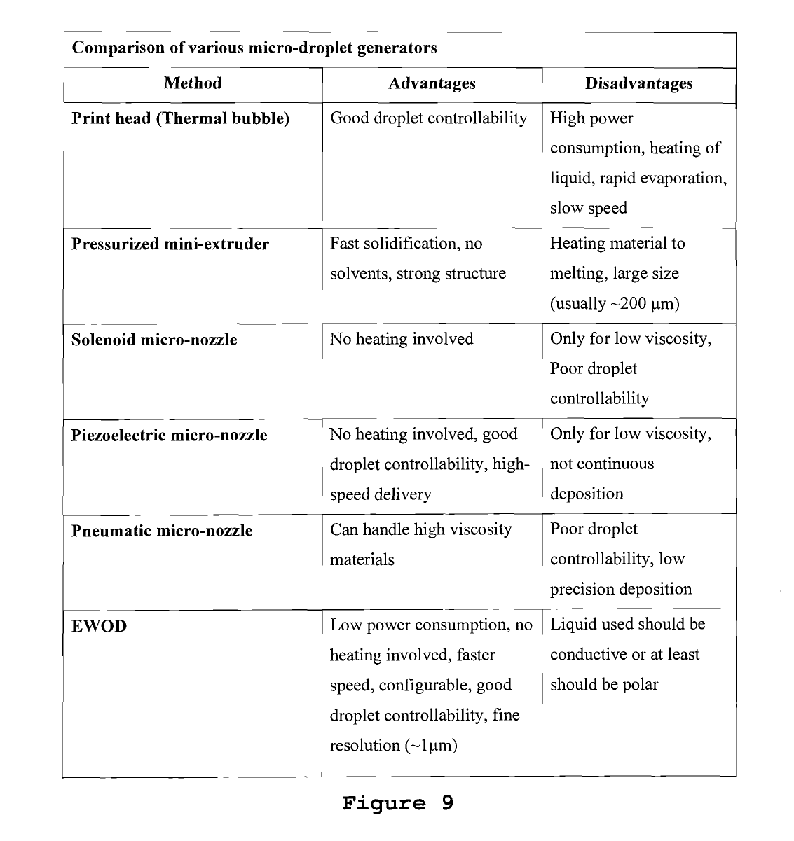

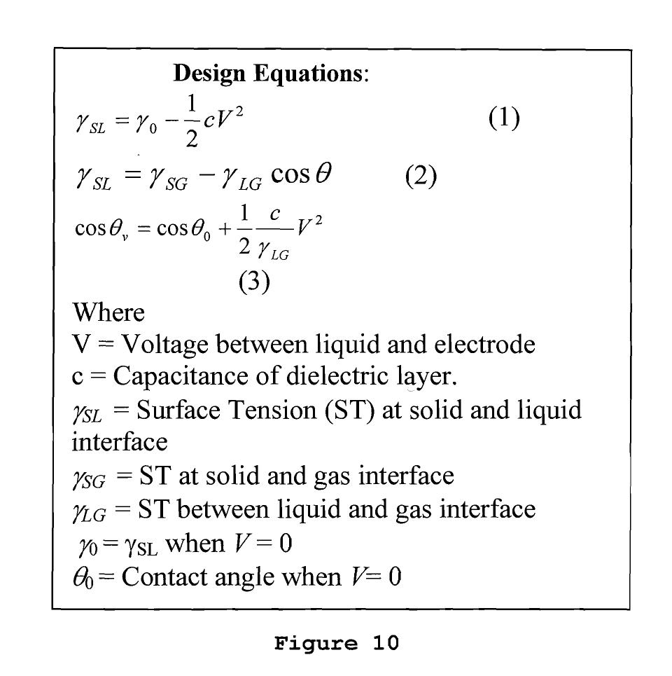

Vertical actuation of droplets ranging in size between nanoliter-sized and microliter-sized was accomplished (FIG. 8). A Teflon-coated platinum wire acted as a grounding connection to the droplet, allowing X-Y axis actuation in the plane of an electrode array. The entire system was immersed in silicone oil to prevent chemicals from adsorbing on the Teflon-coated wire or other transport surfaces. The top piece consisted of an ITO-coated slide attached to a microarray slide. The microarray slide was placed at the bottom of the top layer and faced the drop (see FIG. 8). The bottom plate was fabricated and inserted into a spacer block. The platinum wire strung across the spacer block formed by the ground electrode, and the space between was filled with silicone oil. In operation, a voltage was applied to the ITO layer on the top piece, relative to the ground wire. The ground wire “focused” the electric field, allowing for a significant charge to build up in the droplet. This caused the droplet to be attracted (actuated) vertically upward in the Z-direction to the top microarray plate. The drop was propelled to the top plate and it adhered to the top plate.

The performance of the printing system depicted in part in FIG. 8 has been assessed over a wide range of droplet compositions, volumes ranging over hundreds of nanoliters, variable system dimensions, and different oil and droplet viscosities. A minimum Z-axis actuation voltage of 40-50V was used for these tests. By contrast, aperture-based systems required upwards of 1000V for Z-axis actuation. The actuation voltage for silicone oil droplets at a gap height of 590 μm ranged from 40 to 50V and was almost independent of the drop volume in the 250 nl-700 nl range, and for oil viscosity from 2 to 50 cSt. Furthermore, the actuation voltage was the same for droplets of 20×SSC buffer containing 3M NaCl and 0.3M sodium citrate, over a range of dilutions, and also for deionized water droplets. Further, Z-axis actuation of DNA, and subsequent binding on derivatized glass substrates using an actuation voltage of 40 V was accomplished. The DNA was tagged with Cye5 dye in 1×SSC buffer solution, and 500 nl of this fluid was stamped using the vertical electrostatic method.

### Example 4

### Tissue Construct Manufacture

H9c2 myoblasts are used. H9c2 myoblasts are maintained in DMEM containing 10% FBS and 1% Penicillin-Streptomycin. Upon reaching confluence, and in the presence of low serum concentrations and differentiating growth factors (e.g., bFGF, IGF-1, all-trans-retinoic acid, etc.), H9c2 myoblasts differentiate into cardiac myocytes and eventually fuse to form myotubes. While not contracting spontaneously, the differentiated/fused cells can contract in response to pacing and express numerous cardiomyocyte-like electrophysiological properties, thus demonstrating their histiotypic phenotype.

During the EWOD printing process (FIG. 4), H9c2 cells can be admixed to the test scaffold solution (e.g., chitosan, etc.) at a concentration of 10⁷ cells/ml, to ascertain sufficient cell density (about at least 10 cells/μl), which will facilitate myoblast fusion. Another reservoir contains appropriate growth factor solutions (e.g., bFGF), which also are admixed during the EWOD printing process to yield a growth factor concentration of 50 ng/ml. After the scaffold of desired complexity (for example, geometry (e.g. 10 mm diameter, 1 mm thickness), preferred porosity and composition) is formed/polymerized, the cell- and growth factor-containing scaffolds are transferred into 6 well plates for further culture, biochemical analysis and microscopic observation.

Initially, cell proliferation is assessed every other day using the Alamar blue assay. In select scaffolds, the viability of the cells in the scaffolds is evaluated by confocal fluorescence microscopy using the Live-Dead Assay. Upon reaching confluence (as verified by phase contrast microscopy), differentiation of the myoblasts into myocytes and fusion into myotubes is induced, for example, by reducing the serum contents to 1% and addition of IGF-1 (10 ng/ml) and/or all-trans-retinoic acid (10 nM). Constructs containing differentiated myocytes/myotubes are stimulated electrically. One of skill in the art will be able to empirically determine the optimal pulse parameters (e.g., duration, height, pacing frequency). Electrical activity is monitored using the Microelectrode Array System from Multi Channel Systems (Reutlingen, Germany). Contraction is monitored optically using video graphic analysis software.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. An apparatus for engineering tissue by actuating droplets by electrowetting, comprising:
a. at least four microarray print heads, wherein each of the at least four microarray print heads comprises:
(i) a first conductive layer comprising an array of control electrodes covered by a first hydrophobic insulator surface; and
(ii) a second conductive layer comprising a second conductive layer surface facing the first hydrophobic surface, the second conductive layer spaced from the first conductive layer to define a gap there between, and having an actuation voltage thereon of 20 to 100 volts; and
(iii) a wire traction system comprising at least one conductive elongate wire element disposed in the gap between the first and second conductive layers and comprising a second hydrophobic surface, and having a voltage thereon less than the second conductive layer actuation voltage; and
(iv) a voltage source communicating with the second conductive layer and the elongate wire element that provides an actuation voltage to the second conductive layer of 20 to 100 volts, wherein the droplet is caused to move along a pathway extending around the conductive elongate wire element and from the first conductive layer towards the second conductive layer; and
b. wherein one of the at least four microarray print heads actuates droplets comprising a hydrogel; and
wherein one of the at least four microarray print heads actuates droplets comprising a crosslinker; and
d. wherein one of the at least four microarray print heads actuates droplets comprising a cell suspension; and
e. wherein one of the at least four microarray print heads actuates droplets comprising a growth factor.

2. An apparatus for engineering tissue by manipulating droplets, comprising:
a. at least four microarray print heads, wherein each of the at least four microarray print heads comprises:
(i) a substrate comprising a substrate surface; and
(ii) an array of drive electrodes disposed on the substrate surface; and
(iii) a dedicated array of reference elements settable to a common reference potential and disposed in at least substantially co-planar relation to the electrode array, wherein the array of reference elements is electrically and physically distinct from the drive electrode array and further wherein each drive electrode is adjacent to at least one of the reference elements; and
(iv) a dielectric layer disposed on the substrate surface to cover the drive electrodes; and
(v) an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes; and
b. wherein one of the at least four microarray print heads actuates droplets comprising a hydrogel; and
c. wherein one of the at least four microarray print heads actuates droplets comprising a crosslinker; and
d. wherein one of the at least four microarray print heads actuates droplets comprising a cell suspension; and
e. wherein one of the at least four microarray print heads actuates droplets comprising a growth factor.

3. An apparatus for engineering tissue by manipulating droplets, comprising:
a. at least four microarray print heads, wherein each of the at least four microarray print heads comprises:
(i) a substrate comprising a substrate surface;
(ii) an array of electrodes disposed in at least substantially co-planar relation on the substrate surface, wherein the array of electrodes comprises drive electrodes and dedicated reference electrodes; and
(iii) a dielectric layer disposed on the substrate surface and covering the array of electrodes; and
(iv) an electrode selector for dynamically creating a sequence of electrode pairs, each electrode pair comprising a selected one of the drive electrodes biased to a first voltage and a selected one of the reference electrodes disposed adjacent to the selected drive electrode and biased to a second voltage less than the first voltage, whereby a droplet disposed on the substrate surface moves along a desired path running between the electrode pairs created by the electrode selector; and
(v) whereby manipulation of the droplet is accomplished by electrowetting actuation wherein the droplet overlaps a selected one of the drive electrodes and a selected one of the reference electrodes continuously.
b. wherein one of the at least four microarray print heads actuates droplets comprising a hydrogel; and
c. wherein one of the at least four microarray print heads actuates droplets comprising a crosslinker; and
d. wherein one of the at least four microarray print heads actuates droplets comprising a cell suspension; and
e. wherein one of the at least four microarray print heads actuates droplets comprising a growth factor.

4. A method of tissue engineering by actuating droplets by electrowetting, comprising the steps of:
a. horizontally actuating a first group of droplets comprising a hydrogel to position each droplet of the first group of droplets at a discrete target location on a microfluidic chip, and
b. vertically actuating the first group of droplets to deposit the first group of droplets onto a tissue growth surface; and
c. horizontally actuating a second group of droplets comprising a crosslinker to position each droplet of the second group of droplets at a discrete target location on a microfluidic chip; and
d. vertically actuating the second group of droplets to deposit the second group of droplets onto the tissue growth surface; and
e. horizontally actuating a third group of droplets comprising a cell suspension to position each droplet of the third group of droplets at a discrete target location on a microfluidic chip; and
vertically actuating the third group of droplets to deposit the third group of droplets onto the tissue growth surface; and
g. horizontally actuating a fourth group of droplets comprising a growth factor to position each droplet of the fourth group of droplets at a discrete target location on a microfluidic chip; and
h. vertically actuating the fourth group of droplets to deposit the fourth group of droplets onto the tissue growth surface.

5. The method of claim 4, wherein steps (a) through (h) are repeated to deposit a subsequent layer of droplets onto the tissue growth surface.
